(19) United States
(12) Patent Application Publication
(10) Pub. No.: US 2007/0128591 A1
(43) Pub. Date: Jun. 7, 2007

(54) Methods for diagnosing and treating diseases and conditions of the digestive system and cancer

(76) Inventors: Mark C. Fishman, Newton Center, MA (US); Alan N. Mayer, Brookline, MA (US)

Correspondence Address: CLARK & ELBING LLP, 101 FEDERAL STREET, BOSTON, MA, 02110, US

(21) Appl. No.: 10/483,539

(22) PCT Filed: Jul. 17, 2002
(86) PCT No.: PCT/US02/22904

§ 371 Date: Nov. 5, 2004

Related U.S. Application Data
(60) Provisional application No. 60/306,319, filed on Jul. 17, 2001.

(51) Int. Cl. C12Q 1/68

(52) U.S. Cl. 435/6

(57) ABSTRACT

The invention provides methods of diagnosing diseases and conditions of the digestive system and cancer, methods for identifying compounds that can be used to treat or to prevent such diseases and conditions, and methods of using these compounds to treat or to prevent such diseases and conditions. Also provided in the invention are animal model systems that can be used in screening methods.

### FIELD OF THE INVENTION

This invention relates to methods for diagnosing and treating diseases of the digestive system and cancer.

### BACKGROUND OF THE INVENTION

The cells that line the digestive organs, such as the intestine, pancreas, and liver, arise from a part of the early embryo called the endoderm. The endodermal cells undergo defined movements and changes in cell shape that ultimately lead to the formation of highly organized structures that collectively constitute mature, functioning organs. The individual steps that lead to organ formation have been described by microscopic analysis of developing embryos, but the molecules that are responsible for guiding these steps are largely unknown.

The zebrafish, Danio rerio, is a convenient organism to use in genetic analysis of development. In addition to having a short generation time and being fecund, it has an accessible and transparent embryo, allowing direct observation of organ function from the earliest stages of development.

### SUMMARY OF THE INVENTION

The invention provides diagnostic, drug screening, and therapeutic methods that are based on the observation that a mutation in a zebrafish gene, designated nil per os (npo), which is Latin for “nothing by mouth,” leads to abnormal digestive organ growth and development.

In a first aspect, the invention provides a method of determining whether a test subject (e.g., a mammal, such as a human) has or is at risk of developing a disease or condition related to an npo protein (e.g., a disease or condition of a digestive organ (e.g., the intestine, liver, bile duct, pancreas, stomach, gall bladder, or esophagus), or cancer). This method involves analyzing a nucleic acid molecule of a sample from the test subject to determine whether the test subject has a mutation (e.g., the npo mutation; see below) in a gene encoding the protein. The presence of a mutation indicates that the test subject has or is at risk of developing a disease related to npo. This method can also involve the step of using nucleic acid molecule primers specific for a gene encoding an npo protein for nucleic acid molecule amplification of the gene by the polymerase chain reaction. It can further involve sequencing a nucleic acid molecule encoding an npo protein from a test subject.

In a second aspect, the invention provides a method for identifying a compound that can be used to treat or to prevent a disease or condition of the digestive system or cancer. This method involves contacting an organism (e.g., a zebrafish) having a mutation (e.g., the nil per os mutation) in a gene encoding a nil per os protein and having a phenotype characteristic of such a disease or condition with the compound, and determining the effect of the compound on the phenotype. Detection of an improvement in the phenotype indicates the identification of a compound that can be used to treat or to prevent the disease or condition.

In a third aspect, the invention provides a method of treating or preventing a disease or condition of the digestive system or cancer in a patient (e.g., a patient having a mutation (e.g., the nil per os mutation) in a gene encoding a nil per os protein), involving administering to the patient a compound identified using the method described above. Also included in the invention is the use of such compounds in the treatment or prevention of such diseases or conditions, as well as the use of these compounds in the preparation of a medicament for such treatment or prevention.

In a fourth aspect, the invention provides a method of treating or preventing a disease or condition of the digestive system or cancer in a patient. This method involves administering to the patient a functional nil per os protein or a nucleic acid molecule (in, e.g., an expression vector) encoding the protein. Also included in the invention is the use of such proteins or nucleic acid molecules in the treatment or prevention of such diseases or conditions, as well as the use of these proteins or nucleic acid molecules in the preparation of a medicament for such treatment or prevention.

In a fifth aspect, the invention includes a substantially pure nil per os polypeptide (e.g., a zebrafish or a human npo polypeptide) or a fragment thereof This polypeptide can include or consist essentially of, for example, an amino acid sequence that is substantially identical to the amino acid sequence of SEQ ID NOs:3 or 5. The encoded polypeptide can include RNA recognition motifs (RRMs) and bind RNA, as is discussed further below.

In a sixth aspect, the invention provides a substantially pure nucleic acid molecule (e.g., a DNA molecule) including a sequence encoding a nil per os polypeptide (e.g., a zebrafish or a human npo polypeptide) or a fragment thereof. This nucleic acid molecule can encode a polypeptide including or consisting essentially of an amino sequence that is substantially identical to the amino acid sequence of SEQ ID NOs:3 or 5. The encoded polypeptide can include RNA recognition motifs (RRMs) and bind RNA, as is discussed further below.

In a seventh aspect, the invention provides a vector including the nucleic acid molecule described above.

In an eighth aspect, the invention includes a cell including the vector described above.

In a ninth aspect, the invention provides a non-human transgenic animal (e.g., a zebrafish or a mouse) including the nucleic acid molecule described above.

In a tenth aspect, the invention provides a non-human animal having a knockout mutation in one or both alleles encoding a nil per os polypeptide.

In an eleventh aspect, the invention includes a cell from the non-human knockout animal described above.

In a twelfth aspect, the invention includes a non-human transgenic animal (e.g., a zebrafish) including a nucleic acid molecule encoding a mutant nil per os polypeptide, e.g., a polypeptide having the nil per os mutation.

In a thirteenth aspect, the invention provides an antibody that specifically binds to a nil per os polypeptide.

In a fourteenth aspect, the invention provides a method of modulating nil per os protein activity by administration of an RNA that stimulates or inhibits this activity. Also included in the invention is the use of such an RNA molecule to stimulate or to inhibit this activity, as well as the use of this RNA molecule in the preparation of a medicament for such stimulation or inhibition.

In a fifteenth aspect, the invention provides a method of identifying a stem cell of the gastrointestinal tract, which involves analyzing a pool of candidate cells for expression of nil per os. Cells that express nil per os can then, optionally, be removed from the original pool of candidate cells.

By “polypeptide” or “polypeptide fragment” is meant a chain of two or more amino acids, regardless of any post-translational modification (e.g., glycosylation or phosphorylation), constituting all or part of a naturally or non-naturally occurring polypeptide. By “post-translational modification” is meant any change to a polypeptide or polypeptide fragment during or after synthesis. Post-translational modifications can be produced naturally (such as during synthesis within a cell) or generated artificially (such as by recombinant or chemical means). A “protein” can be made up of one or more polypeptides.

By “nil per os protein,” “npo protein,” “nil per os polypeptide,” or “npo polypeptide” is meant a polypeptide that has at least 45%, preferably at least 60%, more preferably at least 75%, 80%, or 85%, and most preferably at least 90% or 95% amino acid sequence identity to the sequence of a human (SEQ ID NO:5) or a zebrafish (SEQ ID NO:3) nil per os polypeptide. Polypeptide products from splice variants of nil per os gene sequences and nil per os genes containing mutations are also included in this definition. A nil per os polypeptide as defined herein plays a role in digestive organ development, modeling, and function. It can be used as a marker of diseases and conditions of the digestive system, digestive organs, or cancer.

By a “nil per os nucleic acid molecule” or “npo nucleic acid molecule” is meant a nucleic acid molecule, such as a genomic DNA, cDNA, or RNA (e.g., mRNA) molecule, that encodes a nil per os protein (e.g., a human (encoded by SEQ ID NO:4) or a zebrafish (encoded by SEQ ID NOs:1 or 2) nil per os protein), a nil per os polypeptide, or a portion thereof, as defined above. A mutation in a nil per os nucleic acid molecule can be characterized, for example, by a tyrosine codon to stop codon change (TAT to TAA) in the codon for amino acid 221. In addition to this zebrafish nil per os mutation (hereinafter referred to as “the nil per os mutation”), the invention includes any mutation that results in aberrant nil per os protein production or function, including, only as examples, null mutations and additional mutations causing truncations.

The term “identity” is used herein to describe the relationship of the sequence of a particular nucleic acid molecule or polypeptide to the sequence of a reference molecule of the same type. For example, if a polypeptide or a nucleic acid molecule has the same amino acid or nucleotide residue at a given position, compared to a reference molecule to which it is aligned, there is said to be “identity” at that position. The level of sequence identity of a nucleic acid molecule or a polypeptide to a reference molecule is typically measured using sequence analysis software with the default parameters specified therein, such as the introduction of gaps to achieve an optimal alignment (e.g., Sequence Analysis Software Package of the Genetics Computer Group, University of Wisconsin Biotechnology Center, 1710 University Avenue, Madison, WI 53705, BLAST, or PILEUP/PRETTYBOX programs). These software programs match identical or similar sequences by assigning degrees of identity to various substitutions, deletions, or other modifications. Conservative substitutions typically include substitutions within the following groups: glycine, alanine, valine, isoleucine, and leucine; aspartic acid, glutamic acid, asparagine, and glutamine; serine and threonine; lysine and arginine; and phenylalanine and tyrosine.

A nucleic acid molecule or polypeptide is said to be “substantially identical” to a reference molecule if it exhibits, over its entire length, at least 51%, preferably at least 55%, 60%, or 65%, and most preferably 75%, 85%, 90%, or 95% identity to the sequence of the reference molecule. For polypeptides, the length of comparison sequences is at least 16 amino acids, preferably at least 20 amino acids, more preferably at least 25 amino acids, and most preferably at least 35 amino acids. For nucleic acid molecules, the length of comparison sequences is at least 50 nucleotides, preferably at least 60 nucleotides, more preferably at least 75 nucleotides, and most preferably at least 110 nucleotides. Of course, the length of comparison can be any length up to and including full length.

A nil per os nucleic acid molecule or a nil per os polypeptide is “analyzed” or subject to “analysis” if a test procedure is carried out on it that allows the determination of its biological activity or whether it is wild type or mutated. For example, one can analyze the nil per os genes of an animal (e.g., a human or a zebrafish) by amplifying genomic DNA of the animal using the polyrnerase chain reaction, and then determining whether the amplified DNA contains a mutation, for example, the nil per os mutation, by, e.g., nucleotide sequence or restriction fragment analysis.

By “probe” or “primer” is meant a single-stranded DNA or RNA molecule of defined sequence that can base pair to a second DNA or RNA molecule that contains a complementary sequence (a “target”). The stability of the resulting hybrid depends upon the extent of the base pairing that occurs. This stability is affected by parameters such as the degree of complementarity between the probe and target molecule, and the degree of stringency of the hybridization conditions. The degree of hybridization stringency is affected by parameters such as the temperature, salt concentration, and concentration of organic molecules, such as formamide, and is determined by methods that are well known to those skilled in the art. Probes or primers specific for nil per os nucleic acid molecules, preferably, have greater than 45% sequence identity, more preferably at least 55-75% sequence identity, still more preferably at least 75-85% sequence identity, yet more preferably at least 85-99% sequence identity, and most preferably 100% sequence identity to the sequences of human (SEQ ID NO:4) or zebrafish (SEQ ID NOs:1 and 2) nil per os genes.

Probes can be detectably labeled, either radioactively or non-radioactively, by methods that are well known to those skilled in the art. Probes can be used for methods involving nucleic acid hybridization, such as nucleic acid sequencing, nucleic acid amplification by the polymerase chain reaction, single stranded conformational polymorphism (SSCP) analysis, restriction fragment polymorphism (RFLP) analysis, Southern hybridization, northern hybridization, in situ hybridization, electrophoretic mobility shift assay (EMSA), and other methods that are well known to those skilled in the art.

A molecule, e.g., an oligonucleotide probe or primer, a gene or fragment thereof, a cDNA molecule, a polypeptide, or an antibody, can be said to be “detectably-labeled” if it is marked in such a way that its presence can be directly identified in a sample. Methods for detectably labeling molecules are well known in the art and include, without limitation, radioactive labeling (e.g., with an isotope, such as ³²P or ³⁵S) and nonradioactive labeling (e.g., with a fluorescent label, such as fluorescein).

By “substantially pure” is meant a polypeptide or polynucleotide (or a fragment thereof) that has been separated from the proteins and organic molecules that naturally accompany it. Typically, a polypeptide or polynucleotide is substantially pure when it is at least 60%, by weight, free from the proteins and naturally occurring organic molecules with which it is naturally associated. Preferably, the polypeptide or polynucleotide is a nil per os polypeptide or polynucleotide that is at least 75%, more preferably at least 90%, and most preferably at least 99%, by weight, pure. A substantially pure nil per os polypeptide can be obtained, for example, by extraction from a natural source (e.g., an isolated digestive organ), by expression of a recombinant nucleic acid molecule encoding a nil per os polypeptide, or by chemical synthesis. Purity can be measured by any appropriate method, e.g., by column chromatography, polyacrylamide gel electrophoresis, or HPLC analysis. The polynucleotide can also be “isolated,” which means that it is separated from flanking nucleotide sequences that naturally accompany it in the genome. An isolated polynucleotide sequence can include coding sequences only or, alternatively, can also include promoter and other regulatory sequences associated with the coding sequences.

A polypeptide is substantially free of naturally associated components when it is separated from those proteins and organic molecules that accompany it in its natural state. Thus, a protein that is chemically synthesized or produced in a cellular system that is different from the cell in which it is naturally produced is substantially free from its naturally associated components. Accordingly, substantially pure polypeptides not only include those that are derived from eukaryotic organisms, but also those synthesized in E. coli, other prokaryotes, or in other such systems.

An antibody is said to “specifically bind” to a polypeptide if it recognizes and binds to the polypeptide (e.g., a nil per os polypeptide), but does not substantially recognize and bind to other molecules (e.g., non-nil per os related polypeptides) in a sample, e.g., a biological sample, which naturally includes the polypeptide.

By “high stringency conditions” is meant conditions that allow hybridization comparable with the hybridization that occurs using a DNA probe of at least 100, e.g., 200, 350, or 500, nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, 1× Denhardt's solution, 10% dextran sulfate, and 0.1% SDS, at a temperature of 42° C. (These are typical conditions for high stringency northern or Southern hybridizations.) High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually 16 nucleotides or longer for PCR or sequencing, and 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference.

By “sample” is meant a tissue biopsy, amniotic fluid, cell, blood, serum, urine, stool, or other specimen obtained from a patient or a test subject. The sample can be analyzed to detect a mutation in a nil per os gene, or expression levels of a nil per os gene, by methods that are known in the art. For example, methods such as sequencing, single-strand conformational polymorphism (SSCP) analysis, or restriction fragment length polymorphism (RFLP) analysis of PCR products derived from a patient sample can be used to detect a mutation in a nil per os gene; ELISA and other immunoassays can be used to measure levels of a nil per os polypeptide; and PCR can be used to measure the level of a nil per os nucleic acid molecule.

By “nil per os-related disease,” “npo-related disease,” “nil per os-related condition,” or “npo-related condition” is meant a disease or condition that results from inappropriately high or low expression of a nil per os gene, or a mutation in a nil per os gene (including control sequences, such as promoters) that alters the biological activity of a nil per os nucleic acid molecule or polypeptide. Nil per os-related diseases and conditions can arise in any tissue in which nil per os is expressed during prenatal or post-natal life. Nil per os-related diseases and conditions can include diseases or conditions of a digestive organ (e.g., intestine, liver, bile duct, pancreas, gall bladder, stomach, or esophagus) or cancer.

The invention provides several advantages. For example, using the diagnostic methods of the invention it is possible to detect an increased likelihood of diseases or conditions of the digestive system or cancer in a patient, so that appropriate intervention can be instituted before any symptoms occur. This may be useful, for example, with patients in high-risk groups for such diseases or conditions. Also, the diagnostic methods of the invention facilitate determination of the etiology of an existing disease or condition of the digestive system or cancer in a patient, so that an appropriate approach to treatment can be selected. In addition, the screening methods of the invention can be used to identify compounds that can be used to treat or to prevent these diseases or conditions. The invention can also be used to treat diseases or conditions (e.g., digestive organ failure) for which, prior to the invention, the only treatment was organ transplantation, which is limited by the availability of donor organs and the possibility of organ rejection.

Other features and advantages of the invention will be apparent from the following detailed description, the drawings, and the claims.

### BRIEF DESCRIPTION OF THE DRAWINGS

FIGS. 1A-1G show the phenotypic differences between wild type (panels A, C, E, and G) and npo mutant (panels B, D, F, and H) zebrafish. Additional details of this analysis are provided below.

FIG. 2 is a schematic representation of the region of the zebrafish genome in which the npo gene (RRM) is located. The mutation that gives rise to the npo phenotype, which results in the codon for amino acid 221 being converted from that for tyrosine to a stop codon, is illustrated at the bottom of the figure.

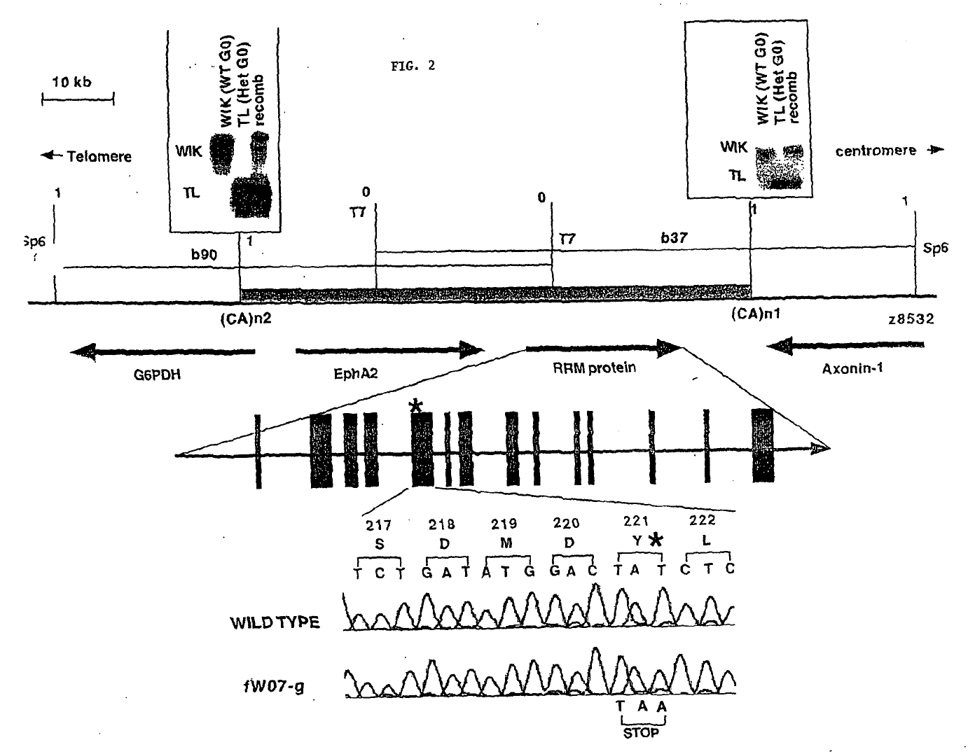

FIG. 3 is a schematic illustration of the RRM domains of the npo protein, with the npo mutation indicated by an asterisk, as well as an alignment of the sequences of zebrafish, human, Drosophila, C. elegans, S. cerevisiae, and Arabidopsis npo. The premature stop codon associated with the npo mutation is, again, indicated by an asterisk.

### DETAILED DESCRIPTION

The invention provides methods of diagnosing and treating diseases and conditions of the digestive system (e.g., the intestine (large or small), liver, pancreas, stomach, gall bladder, or esophagus) or cancer, screening methods for identifying compounds that can be used to treat or to prevent such diseases and conditions, and methods of treating or preventing such diseases and conditions using such compounds. In particular, we have discovered that a mutation (the nil per os (npo) mutation) in a zebrafish gene leads to a phenotype in zebrafish characterized by abnormal digestive organ growth and development. Thus, the diagnostic methods of the invention involve detection of mutations in genes encoding nil per os proteins, while the compound identification methods involve screening for compounds that affect the phenotype of organisms having mutations in genes encoding such proteins or other models of digestive tract diseases and conditions. Compounds identified in this manner, as well as npo genes and proteins themselves, can be used in methods to treat or to prevent digestive tract diseases and conditions. Compounds, antisense molecules, and antibodies that are found to inhibit npo function can also be used to prevent or to treat cancer.

Also provided in the invention are animal model systems (e.g., zebrafish having mutations (e.g., the nil per os mutation) in genes encoding the nil per os protein, or mice (or other animals) having such mutations) that can be used in the screening methods mentioned above, as well as the nil per os protein, and genes encoding this protein. The invention also includes genes encoding mutant zebrafish nil per os proteins (e.g., genes having the nil per os mutation) and proteins encoded by these genes. Antibodies that specifically bind to these proteins (wild type or mutant) are also included in the invention.

The diagnostic, screening, and therapeutic methods of the invention, as well as the animal model systems, proteins, and genes of the invention, are described further, as follows.

Diagnostic Methods

Nucleic acid molecules encoding the nil per os protein, as well as polypeptides encoded by these nucleic acid molecules and antibodies specific for these polypeptides, can be used in methods to diagnose or to monitor diseases and conditions involving mutations in, or inappropriate expression of, genes encoding this protein. As discussed above, the nil per os mutation in zebrafish is characterized by a phenotype in which there is abnormal digestive organ growth and development. Thus, detection of abnormalities in nil per os genes or in their expression can be used in methods to diagnose, or to monitor treatment or development of, diseases or conditions of digestive organs. Also, nil per os plays a role in cell growth control. Thus, detection of abnormalities in this gene (or the protein it encodes) can be used in the diagnosis of cancer, as well as in monitoring cancer treatment.

The diagnostic methods of the invention can be used, for example, with patients that have a disease or condition of the digestive tract or cancer, in an effort to determine its etiology and, thus, to facilitate selection of an appropriate course of treatment. The diagnostic methods can also be used with patients who have not yet developed, but who are at risk of developing, such a disease or condition, or with patients that are at an early stage of developing such a disease or condition. Also, the diagnostic methods of the invention can be used in prenatal genetic screening, for example, to identify parents who may be carriers of a recessive mutation in a gene encoding a nil per os protein.

Diseases or conditions of the digestive tract that can be diagnosed (and treated) using the methods of the invention include any diseases or conditions that affect a digestive organ, such as the intestine (large or small), liver, biliary tract, pancreas, stomach, gall bladder, or esophagus. For example, the methods can be used to diagnose (or to treat) digestive organ failure (e.g., liver failure), inflammatory bowel disease (e.g., Crohn's disease or ulcerative colitis), diverticular disease (e.g., diverticulitis or diverticulosis), malabsorption, steatorrhea, ischemic bowel disease, irritable bowel syndrome, celiac disease, colitis, hepatitis (e.g., autoimmune hepatitis or hepatitis A, B, C, D, E, or G), cirrhosis, fatty liver, gastritis (acute and chronic), gastric ulcer, hyperplastic gastropathy, peptic ulcer, oral-pharyngeal dysphagia, achalasia, and gastro-esophageal reflux disease.

The methods of the invention can also be used in the diagnosis (and treatment) of cancer, e.g., cancer of the digestive tract. For example, the methods of the invention can be used to diagnose or to treat colon cancer, rectal cancer, liver cancer (e.g., hepatocellular carcinoma), pancreatic cancer (exocrine or islet), cancer of the gall bladder, esophageal cancer, stomach cancer, or bile duct cancer, and these cancers can be, for example, adenocarcinomas, adenomas, carcinoids, or carcinomas.

The methods of the invention can be used to diagnose (or to treat) the disorders described herein in any mammal, for example, in humans, domestic pets, or livestock.

Abnormalities in nil per os that can be detected using the diagnostic methods of the invention include those characterized by, for example, (i) a gene encoding a nil per os protein containing a mutation that results in the production of an abnormal nil per os protein, (ii) an abnormal nil per os polypeptide itself (e.g., a truncated protein), and (iii) a mutation in a gene encoding a nil per os protein that results in production of an abnormal amount of this protein. Detection of such abnormalities can be used in methods to diagnose human diseases or conditions of the digestive tract. Exemplary of the mutations in a nil per os protein is the nil per os mutation, which is described further below.

A mutation in a gene encoding a nil per os protein can be detected in any tissue of a subject, even one in which this protein is not expressed. Because of the possibly limited number of tissues in which these proteins may be expressed, for limited time periods, and because of the possible undesirability of sampling such tissues (e.g., digestive organs) for assays, it may be preferable to detect mutant genes in other, more easily obtained sample types, such as in blood or amniotic fluid samples.

Detection of a mutation in a gene encoding a nil per os protein can be carried out using any standard diagnostic technique. For example, a biological sample obtained from a patient can be analyzed for one or more mutations (e.g., a nil per os mutation) in nucleic acid molecules encoding a nil per os protein using a mismatch detection approach. Generally, this approach involves polymerase chain reaction (PCR) amplification of nucleic acid molecules from a patient sample, followed by identification of a mutation (i.e., a mismatch) by detection of altered hybridization, aberrant electrophoretic gel migration, binding, or cleavage mediated by mismatch binding proteins, or by direct nucleic acid molecule sequencing. Any of these techniques can be used to facilitate detection of a mutant gene encoding a nil per os protein, and each is well known in the art. For instance, examples of these techniques are described by Orita et al. (Proc. Natl. Acad. Sci. U.S.A. 86:2766-2770, 1989) and Sheffield et al. (Proc. Natl. Acad. Sci. U.S.A. 86:232-236, 1989).

As noted above, in addition to facilitating diagnosis of an existing disease or condition, mutation detection assays also provide an opportunity to diagnose a predisposition to disease related to a mutation in a gene encoding a nil per os protein before the onset of symptoms. For example, a patient who is heterozygous for a gene encoding an abnormal nil per os protein (or an abnormal amount thereof) that suppresses normal nil per os biological activity or expression may show no clinical symptoms of a disease related to such proteins, and yet possess a higher than normal probability of developing such disease. Given such a diagnosis, a patient can take precautions to minimize exposure to adverse environmental factors, and can carefully monitor their medical condition, for example, through frequent physical examinations. As mentioned above, this type of diagnostic approach can also be used to detect a mutation in a gene encoding the nil per os protein in prenatal screens.

While it may be preferable to carry out diagnostic methods for detecting a mutation in a gene encoding the nil per os protein using genomic DNA from readily accessible tissues, as noted above, mRNA encoding this protein, or the protein itself, can also be assayed from tissue samples in which it is expressed, and may not be so readily accessible. Expression levels of a gene encoding the nil per os protein in such a tissue sample from a patient can be determined by using any of a number of standard techniques that are well known in the art, including northern blot analysis and quantitative PCR (see, e.g., Ausubel et al., supra; PCR Technology: Principles and Applications for DNA Amplification, H. A. Ehrlich, Ed., Stockton Press, NY; Yap et al. Nucl. Acids. Res. 19:4294, 1991).

In another diagnostic approach of the invention, an immunoassay is used to detect or to monitor the level of a nil per os protein in a biological sample. Polyclonal or monoclonal antibodies specific for the nil per os protein can be used in any standard immunoassay format (e.g., ELISA, Western blot, or RIA; see, e.g., Ausubel et al., supra) to measure polypeptide levels of the nil per os protein. These levels can be compared to levels of the nil per os protein in a sample from an unaffected individual. Detection of a decrease in production of the nil per os protein using this method, for example, may be indicative of a condition or a predisposition to a condition involving insufficient biological activity of the nil per os protein.

Immunohistochemical techniques can also be utilized for detection of the nil per os protein in patient samples. For example, a tissue sample can be obtained from a patient, sectioned, and stained for the presence of the nil per os protein using an anti-nil per os protein antibody and any standard detection system (e.g., one that includes a secondary antibody conjugated to an enzyme, such as horseradish peroxidase). General guidance regarding such techniques can be found in, e.g., Bancroft et al., Theory and Practice of Histological Techniques, Churchill Livingstone, 1982, and Ausubel et al., supra.

Identification of Molecules that can be Used to Treat or to Prevent Diseases or Conditions of the Digestive Tract or Cancer

Identification of a mutation in the gene encoding the nil per os protein as resulting in a phenotype that results in abnormal digestive organ growth and development facilitates the identification of molecules (e.g., small organic or inorganic molecules, peptides, or nucleic acid molecules) that can be used to treat or to prevent diseases or conditions of the digestive system or cancer. The effects of candidate compounds on such diseases or conditions can be investigated using, for example, the zebrafish system. As is mentioned above, the zebrafish, Danio rerio, is a convenient organism to use in the genetic analysis of development. It has an accessible and transparent embryo, allowing direct observation of organ function from the earliest stages of development, and has a short generation time and is fecund. As discussed further below, zebrafish and other animals having a nil per os mutation, which can be used in these methods, are also included in the invention.

In one example of the screening methods of the invention, a zebrafish having a mutation in a gene encoding the nil per os protein (e.g., a zebrafish having the nil per os mutation) is contacted with a candidate compound, and the effect of the compound on the development of a digestive tract abnormality, or on the status of such an existing abnormality, is monitored relative to an untreated, identically mutant control.

After a compound has been shown to have a desired effect in the zebrafish system, it can be tested in other models of digestive tract disease, for example, in mice or other animals having a mutation in a gene encoding the nil per os protein. Alternatively, testing in such animal model systems can be carried out in the absence of zebrafish testing. Compounds of the invention can also be tested in animal models of cancer.

Cell culture-based assays can also be used in the identification of molecules that increase or decrease nil per os levels or biological activity. According to one approach, candidate molecules are added at varying concentrations to the culture medium of cells expressing nil per os mRNA. Nil per os biological activity is then measured using standard techniques. The measurement of biological activity can include the measurement of nil per os protein and nucleic acid molecule levels.

In general, novel drugs for the prevention or treatment of diseases related to mutations in genes encoding the nil per os protein can be identified from large libraries of natural products, synthetic (or semi-synthetic) extracts, and chemical libraries using methods that are well known in the art. Those skilled in the field of drug discovery and development will understand that the precise source of test extracts or compounds is not critical to the screening methods of the invention and that dereplication, or the elimination of replicates or repeats of materials already known for their therapeutic activities for nil per os, can be employed whenever possible.

Candidate compounds to be tested include purified (or substantially purified) molecules or one or more components of a mixture of compounds (e.g., an extract or supernatant obtained from cells; Ausubel et al., supra) and such compounds further include both naturally occurring or artificially derived chemicals and modifications of existing compounds. For example, candidate compounds can be polypeptides, synthesized organic or inorganic molecules, naturally occurring organic or inorganic molecules, nucleic acid molecules, and components thereof.

Numerous sources of naturally occurring candidate compounds are readily available to those skilled in the art. For example, naturally occurring compounds can be found in cell (including plant, fungal, prokaryotic, and animal) extracts, mammalian serum, growth medium in which mammalian cells have been cultured, protein expression libraries, or fermentation broths. In addition, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla.), and PharmaMar, U.S.A. (Cambridge, Mass.). Furthermore, libraries of natural compounds can be produced, if desired, according to methods that are known in the art, e.g., by standard extraction and fractionation.

Artificially derived candidate compounds are also readily available to those skilled in the art. Numerous methods are available for generating random or directed synthesis (e.g., semi-synthesis or total synthesis) of any number of chemical compounds, including, for example, saccharide-, lipid-, peptide-, and nucleic acid molecule-based compounds. In addition, synthetic compound libraries are commercially available from Brandon Associates (Merrimack, N.H.) and Aldrich Chemicals (Milwaukee, Wis.). Libraries of synthetic compounds can also be produced, if desired, according to methods known in the art, e.g., by standard extraction and fractionation. Furthermore, if desired, any library or compound can be readily modified using standard chemical, physical, or biochemical methods.

When a crude extract is found to have an effect on the development or persistence of a digestive tract disease, further fractionation of the positive lead extract can be carried out to isolate chemical constituents responsible for the observed effect. Thus, the goal of the extraction, fractionation, and purification process is the careful characterization and identification of a chemical entity within the crude extract having a desired activity. The same assays described herein for the detection of activities in mixtures of compounds can be used to purify the active component and to test derivatives of these compounds. Methods of fractionation and purification of such heterogeneous extracts are well known in the art. If desired, compounds shown to be useful agents for treatment can be chemically modified according to methods known in the art.

In general, compounds that are found to activate npo expression or activity may be used in the prevention or treatment of diseases or conditions of digestive tract, such as those that are characterized by abnormal growth or development, or organ failure. Compounds that are found to block npo expression or activity may be used to prevent or to treat cancer.

Animal Model Systems

The invention also provides animal model systems for use in carrying out the screening methods described above. Examples of these model systems include zebrafish and other animals, such as mice, that have a mutation (e.g., the nil per os mutation) in a gene encoding the nil per os protein. For example, a zebrafish model that can be used in the invention can include a mutation that results in a lack of nil per os protein production or production of a truncated (e.g., by introduction of a stop codon) or otherwise altered nil per os gene product. As a specific example, a zebrafish having the nil per os mutation can be used (see below).

Treatment or Prevention of Digestive System Diseases or Conditions or Cancer

Compounds identified using the screening methods described above can be used to treat patients that have or are at risk of developing diseases or conditions of the digestive system or cancer. Nucleic acid molecules encoding the nil per os protein, as well as these proteins themselves, can also be used in such methods. Treatment may be required only for a short period of time or may, in some form, be required throughout a patient's lifetime. Any continued need for treatment, however, can be determined using, for example, the diagnostic methods described above. In considering various therapies, it is to be understood that such therapies are, preferably, targeted to the affected or potentially affected organ (e.g., the intestine (large or small), liver, bile duct, pancreas, stomach, gall bladder, or esophagus). Such targeting can be achieved using standard methods.

Treatment or prevention of diseases resulting from a mutated gene encoding the nil per os protein can be accomplished, for example, by modulating the function of a mutant nil per os protein. Treatment can also be accomplished by delivering normal nil per os protein to appropriate cells, altering the levels of normal or mutant nil per os protein, replacing a mutant gene encoding a nil per os protein with a normal gene encoding a nil per os protein, or administering a normal gene encoding a nil per os protein. It is also possible to correct the effects of a defect in a gene encoding a nil per os protein by modifying the physiological pathway (e.g., a signal transduction pathway) in which a nil per os protein participates.

In a patient diagnosed as being heterozygous for a gene encoding a mutant nil per os protein, or as susceptible to such mutations or aberrant nil per os expression (even if those mutations or expression patterns do not yet result in alterations in expression or biological activity of nil per os), any of the therapies described herein can be administered before the occurrence of the disease phenotype. In particular, compounds shown to have an effect on the phenotype of mutants, or to modulate expression of nil per os proteins can be administered to patients diagnosed with potential or actual disease by any standard dosage and route of administration.

Any appropriate route of administration can be employed to administer a compound identified as described above, an npo gene, or an npo protein, according to the invention. For example, administration can be parenteral, intravenous, intra-arterial, subcutaneous, intramuscular, intraventricular, intracapsular, intraspinal, intracisternal, intraperitoneal, intranasal, by aerosol, by suppository, or oral.

A therapeutic compound of the invention can be administered within a pharmaceutically-acceptable diluent, carrier, or excipient, in unit dosage form. Administration can begin before or after the patient is symptomatic. Methods that are well known in the art for making formulations are found, for example, in Remington's Pharmaceutical Sciences (18^{th} edition), ed. A. Gennaro, 1990, Mack Publishing Company, Easton, Pa. Therapeutic formulations can be in the form of liquid solutions or suspensions. Formulations for parenteral administration can, for example, contain excipients, sterile water, or saline; polyalkylene glycols, such as polyethylene glycol; oils of vegetable origin; or hydrogenated napthalenes. Biocompatible, biodegradable lactide polymer, lactide/glycolide copolymer, or polyoxyethylene-polyoxypropylene copolymers can be used to control the release of the compounds. Other potentially useful parenteral delivery systems include ethylene-vinyl acetate copolymer particles, osmotic pumps, implantable infusion systems, and liposomes. For oral administration, formulations can be in the form of tablets or capsules. Formulations for inhalation can contain excipients, for example, lactose, or can be aqueous solutions containing, for example, polyoxyethylene-9-lauryl ether, glycocholate, and deoxycholate, or can be oily solutions for administration in the form of nasal drops or as a gel. Alternatively, intranasal formulations can be in the form of powders or aerosols.

To replace a mutant protein with normal protein, or to add protein to cells that do not express sufficient or normal nil per os protein, it may be necessary to obtain large amounts of pure nil per os protein from cultured cell systems in which the protein is expressed (see, e.g., below). Delivery of the protein to the affected tissue can then be accomplished using appropriate packaging or administration systems.

Gene therapy is another therapeutic approach for preventing or ameliorating diseases caused by nil per os gene defects. Nucleic acid molecules encoding wild type nil per os protein can be delivered to cells that lack sufficient, normal nil per os protein biological activity (e.g., cells carrying mutations (e.g., the nil per os mutation) in nil per os genes). The nucleic acid molecules must be delivered to those cells in a form in which they can be taken up by the cells and so that sufficient levels of protein, to provide effective nil per os protein function, can be produced. Alternatively, for some nil per os mutations, it may be possible to slow the progression of the resulting disease or to modulate nil per os protein activity by introducing another copy of a homologous gene bearing a second mutation in that gene, to alter the mutation, or to use another gene to block any negative effect.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, the full length nil per os gene, or a portion thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the introduction of therapeutic DNA into cells predicted to be subject to diseases involving the nil per os protein. For example, a nil per os nucleic acid molecule or an antisense nucleic acid molecule can be introduced into a cell by lipofection (Felgner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990).

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nil per os protein into a cultivatable cell type ex vivo, after which the cell (or its descendants) are injected into a targeted tissue.

Nil per os cDNA expression for use in gene therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct nil per os expression. The enhancers used can include, without limitation, those that are characterized as tissue- or cell-specific enhancers. Alternatively, if a nil per os genomic clone is used as a therapeutic construct (such clones can be identified by hybridization with nil per os cDNA, as described herein), regulation can be mediated by the cognate regulatory sequences or, if desired, by regulatory sequences derived from a heterologous source, including any of the promoters or regulatory elements described above.

Molecules for effecting antisense-based strategies can be employed to explore nil per os protein gene function, as a basis for therapeutic drug design, as well as to treat npo-associated cancer. These strategies are based on the principle that sequence-specific suppression of gene expression (via transcription or translation) can be achieved by intracellular hybridization between genomic DNA or mRNA and a complementary antisense species. The formation of a hybrid RNA duplex interferes with transcription of the target nil per os protein-encoding genomic DNA molecule, or processing, transport, translation, or stability of the target nil per os mRNA molecule.

Antisense strategies can be delivered by a variety of approaches. For example, antisense oligonucleotides or antisense RNA can be directly administered (e.g., by intravenous injection) to a subject in a form that allows uptake into cells. Alternatively, viral or plasmid vectors that encode antisense RNA (or antisense RNA fragments) can be introduced into a cell in vivo or ex vivo. Antisense effects can be induced by control (sense) sequences; however, the extent of phenotypic changes is highly variable. Phenotypic effects induced by antisense effects are based on changes in criteria such as protein levels, protein activity measurement, and target mRNA levels.

Nil per os gene therapy can also be accomplished by direct administration of antisense nil per os mRNA to a cell that is expected to be adversely affected by the expression of wild type or mutant nil per os protein. The antisense nil per os mRNA can be produced and isolated by any standard technique, but is most readily produced by in vitro transcription using an antisense nil per os cDNA under the control of a high efficiency promoter (e.g., the T7 promoter). Administration of antisense nil per os mRNA to cells can be carried out by any of the methods for direct nucleic acid molecule administration described above.

An alternative strategy for inhibiting nil per os protein function using gene therapy involves intracellular expression of an anti-nil per os protein antibody or a portion of an anti-nil per os protein antibody. For example, the gene (or gene fragment) encoding a monoclonal antibody that specifically binds to a nil per os protein and inhibits its biological activity can be placed under the transcriptional control of a tissue-specific gene regulatory sequence.

Another therapeutic approach included in the invention involves administration of a recombinant nil per os polypeptide, either directly to the site of a potential or actual disease-affected tissue (for example, by injection) or systemically (for example, by any conventional recombinant protein administration technique). The dosage of the nil per os protein depends on a number of factors, including the size and health of the individual patient but, generally, between 0.1 mg and 100 mg, inclusive, is administered per day to an adult in any pharmaceutically acceptable formulation.

As mentioned above, compounds that are found to activate npo expression or activity may be used in the prevention or treatment of diseases or conditions of the digestive tract, such as those that are characterized by abnormal growth or development (see list above), or organ failure. Npo proteins and nucleic acid molecules themselves can be used in these methods as well. For example, compounds that are found to activate npo expression or activity, npo proteins, or npo nucleic acid molecules can be used to treat digestive organ failure (e.g., liver failure), by stimulating the regeneration of a failing digestive organ. Compounds, antisense molecules, or antibodies that block npo expression or activity may be used to prevent or to treat cancer.

An additional therapeutic method of the invention is based on the fact that the npo protein contains several RNA recognition motifs (RRMs) and, thus, likely functions by the RNA-binding activities of at least some of these motifs. In this method, the function of the npo protein is modulated by the administration of RNA molecules that have been identified, using methods such as those described above, as stimulating or inhibiting npo activity, depending on which effect is desired. For example, to stimulate digestive organ growth or development (e.g., to treat or to prevent any of the diseases or conditions mentioned herein, such as organ failure, or to facilitate organ culture), a stimulatory RNA can be administered. In contrast, to treat or to prevent cancer, an inhibitory RNA can be administered. Of course, as is understood in the art, a DNA molecule that is transcribed in vivo to generate such a stimulatory or inhibitory RNA can be administered as well. Administration of these molecules, whether in a vector, such as a viral vector, or not can be carried out using standard methods that are known in the art (see, e.g., above).

In addition to the therapeutic methods described herein, involving administration of npo-modulating compounds, npo proteins, or npo nucleic acids to patients, the invention provides methods of culturing organs in the presence of such molecules. In particular, as is noted above, an npo mutation is associated with abnormal digestive organ growth and development. Thus, culturing digestive organs in the presence of these molecules can be used to promote their growth and development. These organs can be those that are being prepared for transplant from, e.g., an allogeneic or xenogeneic donor, as well as synthetic organs.

Synthesis of Nil Per Os Proteins, Polypeptides, and Polypeptide Fragments

Those skilled in the art of molecular biology will understand that a wide variety of expression systems can be used to produce the recombinant nil per os proteins. As discussed further below, the precise host cell used is not critical to the invention. The nil per os proteins can be produced in a prokaryotic host (e.g., E. coli) or in a eukaryotic host (e.g., S. cerevisiae, insect cells, such as Sf9 cells, or mammalian cells, such as COS-1, NIH 3T3, or HeLa cells). These cells are commercially available from, for example, the American Type Culture Collection, Manassas, Va. (see also Ausubel et al., supra). The method of transformation and the choice of expression vehicle (e.g., expression vector) will depend on the host system selected. Transformation and transfection methods are described, e.g., in Ausubel et al., supra, and expression vehicles can be chosen from those provided, e.g., in Pouwels et al., Cloning Vectors: A Laboratory Manual, 1985, Supp. 1987. Specific examples of expression systems that can be used in the invention are described further as follows.

For protein expression, eukaryotic or prokaryotic expression systems can be generated in which nil per os gene sequences are introduced into a plasmid or other vector, which is then used to transform living cells. Constructs in which full-length nil per os cDNAs, containing the entire open reading frame, inserted in the correct orientation into an expression plasmid can be used for protein expression. Alternatively, portions of nil per os gene sequences, including wild type or mutant nil per os sequences, can be inserted. Prokaryotic and eukaryotic expression systems allow various important functional domains of nil per os proteins to be recovered, if desired, as fusion proteins, and then used for binding, structural, and functional studies, and also for the generation of antibodies.

Typical expression vectors contain promoters that direct synthesis of large amounts of mRNA corresponding to a nucleic acid molecule that has been inserted into the vector. They can also include a eukaryotic or prokaryotic origin of replication, allowing for autonomous replication within a host cell, sequences that confer resistance to an otherwise toxic drug, thus allowing vector-containing cells to be selected in the presence of the drug, and sequences that increase the efficiency with which the synthesized mRNA is translated. Stable long-term vectors can be maintained as freely replicating entities by using regulatory elements of, for example, viruses (e.g., the OriP sequences from the Epstein Barr Virus genome). Cell lines can also be produced that have the vector integrated into genomic DNA of the cells, and, in this manner, the gene product can be produced in the cells on a continuous basis.

Expression of foreign molecules in bacteria, such as Escherichia coli, requires the insertion of a foreign nucleic acid molecule, e.g., a nil per os nucleic acid molecule, into a bacterial expression vector. Such plasmid vectors include several elements required for the propagation of the plasmid in bacteria, and for expression of foreign DNA contained within the plasmid. Propagation of only plasmid-bearing bacteria is achieved by introducing, into the plasmid, a selectable marker-encoding gene that allows plasmid-bearing bacteria to grow in the presence of an otherwise toxic drug. The plasmid also contains a transcriptional promoter capable of directing synthesis of large amounts of mRNA from the foreign DNA. Such promoters can be, but are not necessarily, inducible promoters that initiate transcription upon induction by culture under appropriate conditions (e.g., in the presence of a drug that activates the promoter). The plasmid also, preferably, contains a polylinker to simplify insertion of the gene in the correct orientation within the vector.

Once an appropriate expression vector containing a nil per os gene, or a fragment, fusion, or mutant thereof, is constructed, it can be introduced into an appropriate host cell using a transformation technique, such as, for example, calcium phosphate transfection, DEAE-dextran transfection, electroporation, microinjection, protoplast fusion, or liposome-mediated transfection. Host cells that can be transfected with the vectors of the invention can include, but are not limited to, E. coli or other bacteria, yeast, fungi, insect cells (using, for example, baculoviral vectors for expression), or cells derived from mice, humans, or other animals. Mammalian cells can also be used to express nil per os proteins using a virus expression system (e.g., a vaccinia virus expression system) described, for example, in Ausubel et al., supra.

In vitro expression of nil per os proteins, fusions, polypeptide fragments, or mutants encoded by cloned DNA can also be carried out using the T7 late-promoter expression system. This system depends on the regulated expression of T7 RNA polymerase, an enzyme encoded in the DNA of bacteriophage T7. The T7 RNA polymerase initiates transcription at a specific 23 base pair promoter sequence called the T7 late promoter. Copies of the T7 late promoter are located at several sites on the T7 genome, but none are present in E. coli chromosomal DNA. As a result, in T7-infected E. coli, T7 RNA polymerase catalyzes transcription of viral genes, but not E. coli genes. In this expression system, recombinant E. coli cells are first engineered to carry the gene encoding T7 RNA polymerase next to the lac promoter. In the presence of IPTG, these cells transcribe the T7 polymerase gene at a high rate and synthesize abundant amounts of T7 RNA polymerase. These cells are then transformed with plasmid vectors that carry a copy of the T7 late promoter protein. When IPTG is added to the culture medium containing these transformed E. coli cells, large amounts of T7 RNA polymerase are produced. The polymerase then binds to the T7 late promoter on the plasmid expression vectors, catalyzing transcription of the inserted cDNA at a high rate. Since each E. coli cell contains many copies of the expression vector, large amounts of mRNA corresponding to the cloned cDNA can be produced in this system and the resulting protein can be radioactively labeled.

Plasmid vectors containing late promoters and the corresponding RNA polymerases from related bacteriophages, such as T3, T5, and SP6, can also be used for in vitro production of proteins from cloned DNA. E. coli can also be used for expression using an M13 phage, such as mGPI-2. Furthermore, vectors that contain phage lambda regulatory sequences, or vectors that direct the expression of fusion proteins, for example, a maltose-binding protein fusion protein or a glutathione-S-transferase fusion protein, also can be used for expression in E. coli.

Eukaryotic expression systems are useful for obtaining appropriate post-translational modification of expressed proteins. Transient transfection of a eukaryotic expression plasmid containing a nil per os protein into a eukaryotic host cell allows the transient production of a nil per os protein by the transfected host cell. Nil per os proteins can also be produced by a stably-transfected eukaryotic (e.g., mammalian) cell line. A number of vectors suitable for stable transfection of mammalian cells are available to the public (see, e.g., Pouwels et al., supra), as are methods for constructing lines including such cells (see, e.g., Ausubel et al., supra).

In one example, cDNA encoding a nil per os protein, fusion, mutant, or polypeptide fragment is cloned into an expression vector that includes the dihydrofolate reductase (DHFR) gene. Integration of the plasmid and, therefore, integration of the nil per os protein-encoding gene, into the host cell chromosome is selected for by inclusion of 0.01-300 μM methotrexate in the cell culture medium (Ausubel et al., supra). This dominant selection can be accomplished in most cell types. Recombinant protein expression can be increased by DHFR-mediated amplification of the transfected gene. Methods for selecting cell lines bearing gene amplifications are described in Ausubel et al., supra. These methods generally involve extended culture in medium containing gradually increasing levels of methotrexate. The most commonly used DHFR-containing expression vectors are pCVSEII-DHFR and pAdD26SV(A) (described, for example, in Ausubel et al., supra). The host cells described above or, preferably, a DHFR-deficient CHO cell line (e.g., CHO DHFR- cells, ATCC Accession No. CRL 9096) are among those that are most preferred for DHFR selection of a stably transfected cell line or DHFR-mediated gene amplification.

Another preferred eukaryotic expression system is the baculovirus system using, for example, the vector pBacPAK9, which is available from Clontech (Palo Alto, Calif.). If desired, this system can be used in conjunction with other protein expression techniques, for example, the myc tag approach described by Evan et al. (Molecular and Cellular Biology 5:3610-3616, 1985).

Once a recombinant protein is expressed, it can be isolated from the expressing cells by cell lysis followed by protein purification techniques, such as affinity chromatography. In this example, an anti-nil per os protein antibody, which can be produced by the methods described herein, can be attached to a column and used to isolate the recombinant nil per os proteins. Lysis and fractionation of nil per os protein-harboring cells prior to affinity chromatography can be performed by standard methods (see, e.g., Ausubel et al., supra). Once isolated, the recombinant protein can, if desired, be purified further by, e.g., high performance liquid chromatography (HPLC; e.g., see Fisher, Laboratory Techniques In Biochemistry and Molecular Biology, Work and Burdon, Eds., Elsevier, 1980).

Polypeptides of the invention, particularly short nil per os protein fragments and longer fragments of the N-terminus and C-terminus of the nil per os protein, can also be produced by chemical synthesis (e.g., by the methods described in Solid Phase Peptide Synthesis, 2^{nd} ed., 1984, The Pierce Chemical Co., Rockford, Ill.). These general techniques of polypeptide expression and purification can also be used to produce and isolate useful nil per os protein fragments or analogs, as described herein.

Nil Per Os Protein Fragments

Polypeptide fragments that include various portions of nil per os proteins are useful in identifying the domains of the nil per os protein (e.g., RRMs) that are important for its biological activities, such as protein-protein interactions, transcription, and RNA binding. Methods for generating such fragments are well known in the art (see, for example, Ausubel et al., supra), using the nucleotide sequences provided herein. For example, a nil per os protein fragment can be generated by PCR amplifying a desired nil per os protein nucleic acid molecule fragment using oligonucleotide primers designed based upon nil per os nucleic acid sequences. Preferably, the oligonucleotide primers include unique restriction enzyme sites that facilitate insertion of the amplified fragment into the cloning site of an expression vector (e.g., a mammalian expression vector, see above). This vector can then be introduced into a cell (e.g., a mammalian cell; see above) by artifice, using any of the various techniques that are known in the art, such as those described herein, resulting in the production of a nil per os protein fragment in the cell containing the expression vector. Nil per os protein fragments (e.g., chimeric fusion proteins) can also be used to raise antibodies specific for various regions of the nil per os protein using, for example, the methods described below.

Nil Per Os Protein Antibodies

To prepare polyclonal antibodies, nil per os proteins, fragments of nil per os proteins, or fusion proteins containing defined portions of nil per os proteins can be synthesized in, e.g., bacteria by expression of corresponding DNA sequences contained in a suitable cloning vehicle. Fusion proteins are commonly used as a source of antigen for producing antibodies. Two widely used expression systems for E. coli are lacZ fusions using the pUR series of vectors and trpE fusions using the pATH vectors. The proteins can be purified, coupled to a carrier protein, mixed with Freund's adjuvant to enhance stimulation of the antigenic response in an inoculated animal, and injected into rabbits or other laboratory animals. Alternatively, protein can be isolated from nil per os protein-expressing cultured cells. Following booster injections at bi-weekly intervals, the rabbits or other laboratory animals are then bled and the sera isolated. The sera can be used directly or can be purified prior to use by various methods, including affinity chromatography employing reagents such as Protein A-Sepharose, antigen-Sepharose, and anti-mouse-Ig-Sepharose. The sera can then be used to probe protein extracts from nil per os protein-expressing tissue fractionated by polyacrylamide gel electrophoresis to identify nil per os proteins. Alternatively, synthetic peptides can be made that correspond to antigenic portions of the protein and used to inoculate the animals.

To generate peptide or full-length protein for use in making, for example, nil per os protein-specific antibodies, a nil per os protein coding sequence can be expressed as a C-terminal or N-terminal fusion with glutathione S-transferase (GST; Smith et al., Gene 67:31-40, 1988). The fusion protein can be purified on glutathione-Sepharose beads, eluted with glutathione, cleaved with a protease, such as thrombin or Factor-Xa (at the engineered cleavage site), and purified to the degree required to successfully immunize rabbits. Primary immunizations can be carried out with Freund's complete adjuvant and subsequent immunizations performed with Freund's incomplete adjuvant. Antibody titers can be monitored by Western blot and immunoprecipitation analyses using the protease-cleaved nil per os protein fragment of the GST-nil per os protein. Immune sera can be affinity purified using CNBr-Sepharose-coupled nil per os protein. Antiserum specificity can be determined using a panel of unrelated GST fusion proteins.

Alternatively, monoclonal nil per os protein antibodies can be produced by using, as an antigen, nil per os protein isolated from nil per os protein-expressing cultured cells or nil per os protein isolated from tissues. The cell extracts, or recombinant protein extracts containing nil per os protein, can, for example, be injected with Freund's adjuvant into mice. Several days after being injected, the mouse spleens can be removed, the tissues disaggregated, and the spleen cells suspended in phosphate buffered saline (PBS). The spleen cells serve as a source of lymphocytes, some of which would be producing antibody of the appropriate specificity. These can then be fused with permanently growing myeloma partner cells, and the products of the fusion plated into a number of tissue culture wells in the presence of selective agents, such as hypoxanthine, aminopterine, and thymidine (HAT). The wells can then be screened by ELISA to identify those containing cells making antibodies capable of binding to a nil per os protein, polypeptide fragment, or mutant thereof. These cells can then be re-plated and, after a period of growth, the wells containing these cells can be screened again to identify antibody-producing cells. Several cloning procedures can be carried out until over 90% of the wells contain single clones that are positive for specific antibody production. From this procedure, a stable line of clones that produce the antibody can be established. The monoclonal antibody can then be purified by affinity chromatography using Protein A Sepharose and ion exchange chromatography, as well as variations and combinations of these techniques. Once produced, monoclonal antibodies are also tested for specific nil per os protein recognition by Western blot or immunoprecipitation analysis (see, e.g., Kohler et al., Nature 256:495, 1975; Kohler et al., European Journal of Immunology 6:511, 1976; Kohler et al., European Journal of Immunology 6:292, 1976; Hammerling et al., In Monoclonal Antibodies and T Cell Hybridomas, Elsevier, New York, N.Y., 1981; Ausubel et al., supra).

As an alternate or adjunct immunogen to GST fusion proteins, peptides corresponding to relatively unique hydrophilic regions of the nil per os protein can be generated and coupled to keyhole limpet hemocyanin (KLH) through an introduced C-terminal lysine. Antiserum to each of these peptides can be similarly affinity-purified on peptides conjugated to BSA, and specificity tested by ELISA and Western blotting using peptide conjugates, and by Western blotting and immunoprecipitation using the nil per os protein, for example, expressed as a GST fusion protein.

Antibodies of the invention can be produced using nil per os protein amino acid sequences that do not reside within highly conserved regions, and that appear likely to be antigenic, as analyzed by criteria such as those provided by the Peptide Structure Program (Genetics Computer Group Sequence Analysis Package, Program Manual for the GCG Package, Version 7, 1991) using the algorithm of Jameson et al., CABIOS 4:181, 1988. These fragments can be generated by standard techniques, e.g., by PCR, and cloned into the pGEX expression vector. GST fusion proteins can be expressed in E. coli and purified using a glutathione-agarose affinity matrix (Ausubel et al., supra). To generate rabbit polyclonal antibodies, and to minimize the potential for obtaining antisera that is non-specific, or exhibits low-affinity binding to a nil per os protein, two or three fusions are generated for each protein, and each fusion is injected into at least two rabbits. Antisera are raised by injections in series, preferably including at least three booster injections.

In addition to intact monoclonal and polyclonal anti-nil per os protein antibodies, the invention features various genetically engineered antibodies, humanized antibodies, and antibody fragments, including F(ab′)2, Fab′, Fab, Fv, and sFv fragments. Truncated versions of monoclonal antibodies, for example, can be produced by recombinant methods in which plasmids are generated that express the desired monoclonal antibody fragment(s) in a suitable host. Antibodies can be humanized by methods known in the art, e.g., monoclonal antibodies with a desired binding specificity can be commercially humanized (Scotgene, Scotland; Oxford Molecular, Palo Alto, Calif.). Fully human antibodies, such as those expressed in transgenic animals, are also included in the invention (Green et al., Nature Genetics 7:13-21, 1994).

Ladner (U.S. Pat. Nos. 4,946,778 and 4,704,692) describes methods for preparing single polypeptide chain antibodies. Ward et al., Nature 341:544-546, 1989, describes the preparation of heavy chain variable domains, which they term “single domain antibodies,” and which have high antigen-binding affinities. McCafferty et al., Nature 348:552-554, 1990, shows that complete antibody V domains can be displayed on the surface of fd bacteriophage, that the phage bind specifically to antigen, and that rare phage (one in a million) can be isolated after affinity chromatography. Boss et al., U.S. Pat. No. 4,816,397, describes various methods for producing immunoglobulins, and immunologically functional fragments thereof, that include at least the variable domains of the heavy and light chains in a single host cell. Cabilly et al., U.S. Pat. No. 4,816,567, describes methods for preparing chimeric antibodies.

Use of Nil Per Os Antibodies

Antibodies to nil per os proteins can be used, as noted above, to detect nil per os proteins or to inhibit the biological activities of nil per os proteins. For example, a nucleic acid molecule encoding an antibody or portion of an antibody can be expressed within a cell to inhibit nil per os protein function. In addition, the antibodies can be coupled to compounds, such as radionuclides and liposomes, for diagnostic or therapeutic uses. Antibodies that specifically recognize extracellular domains of nil per os proteins are useful for targeting such attached moieties to cells displaying such nil per os protein domains at their surfaces. Antibodies that inhibit the activity of a nil per os polypeptide described herein can also be useful in preventing or slowing the development of a disease caused by inappropriate expression of a wild type or mutant nil per os gene.

Detection of Nil Per Os Gene Expression

As noted, the antibodies described above can be used to monitor nil per os protein expression. In situ hybridization of RNA can be used to detect the expression of nil per os genes. RNA in situ hybridization techniques rely upon the hybridization of a specifically labeled nucleic acid probe to the cellular RNA in individual cells or tissues. Therefore, RNA in situ hybridization is a powerful approach for studying tissue- and temporal-specific gene expression. In this method, oligonucleotides, cloned DNA fragments, or antisense RNA transcripts of cloned DNA fragments corresponding to unique portions of nil per os genes are used to detect specific mRNA species, e.g., in the tissues of animals, such as mice, at various developmental stages. Other gene expression detection techniques are known to those of skill in the art and can be employed for detection of nil per os gene expression.

Identification of Additional Nil Per Os Genes

Standard techniques, such as the polymerase chain reaction (PCR) and DNA hybridization, can be used to clone nil per os homologues in other species and nil per os-related genes in humans. Nil per os-related genes and homologues can be readily identified using low-stringency DNA hybridization or low-stringency PCR with human nil per os probes or primers. Degenerate primers encoding human nil per os or human nil per os-related amino acid sequences can be used to clone additional nil per os-related genes and homologues by RT-PCR.

Construction of Transgenic Animals and Knockout Animals

Characterization of nil per os genes provides information that allows nil per os knockout animal models to be developed by homologous recombination. Preferably, a nil per os knockout animal is a mammal, most preferably a mouse. Similarly, animal models of nil per os overproduction can be generated by integrating one or more nil per os sequences into the genome of an animal, according to standard transgenic techniques. Moreover, the effect of nil per os mutations (e.g., dominant gene mutations) can be studied using transgenic mice carrying mutated nil per os transgenes or by introducing such mutations into the endogenous nil per os gene, using standard homologous recombination techniques.

A replacement-type targeting vector, which can be used to create a knockout model, can be constructed using an isogenic genomic clone, for example, from a mouse strain such as 129/Sv (Stratagene Inc., LaJolla, Calif). The targeting vector can be introduced into a suitably derived line of embryonic stem (ES) cells by electroporation to generate ES cell lines that carry a profoundly truncated form of a nil per os gene. To generate chimeric founder mice, the targeted cell lines are injected into a mouse blastula-stage embryo. Heterozygous offspring can be interbred to homozygosity. Nil per os knockout mice provide a tool for studying the role of nil per os in embryonic development and in disease. Moreover, such mice provide the means, in vivo, for testing therapeutic compounds for amelioration of diseases or conditions involving nil per os-dependent or a nil per os-affected pathway.

Use of Nil Per Os as a Marker for Stem Cells of the Gastrointestinal Tract

As nil per os is expressed in cells that give rise to gastrointestinal tract organs and tissues during the course of development, it can be used as a marker for stem cells of the gastrointestinal tract. For example, nil per os can be used to identify, sort, or target such stem cells. A pool of candidate cells, for example, can be analyzed for nil per os expression, to facilitate the identification of gastrointestinal tract stem cells, which, based on this identification can be separated from the pool. The isolated stem cells can be used for many purposes that are known to those of skill in this art. For example, the stem cells can be used in the production of new organs, in organ culture, or to fortify damaged or transplanted organs.

Experimental Results

In a genetic screen of chemically mutagenized zebrafish, we identified a mutation that leads to abnormal digestive organ development. Adult male zebrafish (TL strain) were treated with ENU and outcrossed for two generations. Inbred progeny were screened for defects visible on day 4 using dissecting microscopy, and the fW07-g allele showed clearly defective growth of the intestine, liver, and pancreas. The mutant phenotype displays recessive, fully penetrant Mendelian inheritance, as 25% of progeny from heterozygote crosses exhibit the phenotype, and the mutation has bred true through subsequent generations of outcrossing. These observations are consistent with loss of function of a single essential gene. There is no detectable effect of genetic background on the phenotype.

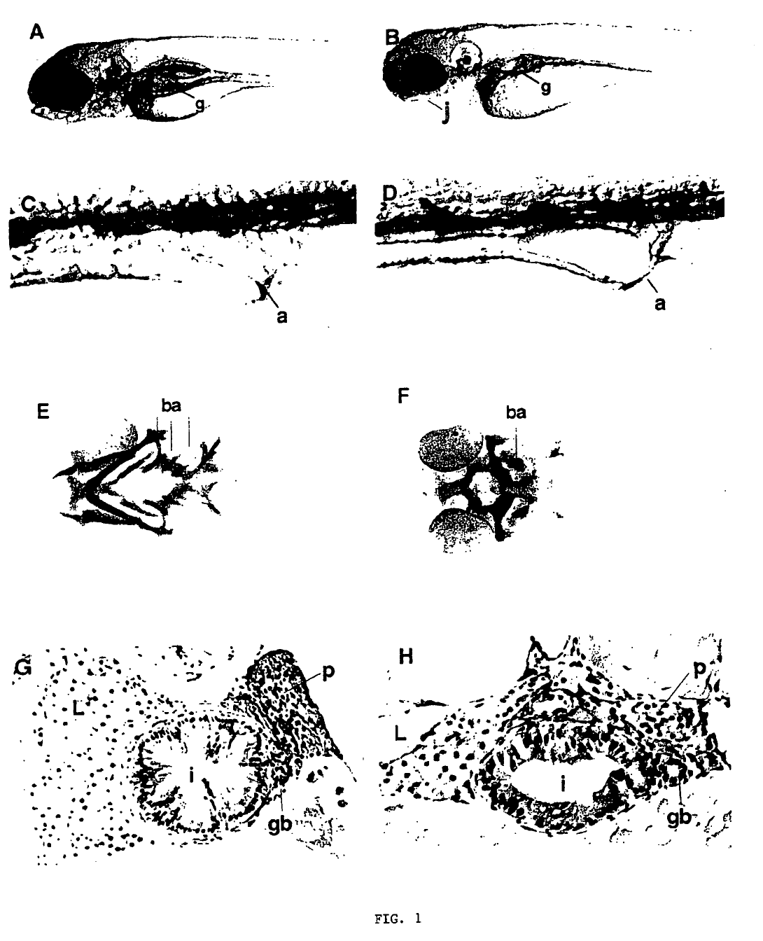

The phenotype is first discernible at about 72 hours post fertilization (hpf), when gut and jaw outgrowth fail to occur. These defects become more pronounced by 96 hpf, when the wild type gut has expanded considerably, but the mutant gut is barely visible (FIGS. 1A and 1B). The mutant anal orifice is obstructed, but the cloaca and the pronephric duct appear to be patent (FIGS. 1C and 1D). The jaw and bronchial arches fail to develop, as seen by alcian blue staining (FIGS. 1E and 1F). Other affected organs include the swim bladder and the pancreas. Anti-insulin immunofluorescence shows that the pancreatic islets retain normal size and architecture, thus indicating that the mutation selectively affects endoderm-derived epithelium. Heart function is normal, and brain, neural tube, kidney, and notocord morphogenesis show no abnormalities in the mutant. The mutant larvae do not eat, presumably due to the structural jaw defect, hence the name nil per os, which, as noted above, is Latin for “nothing by mouth.”

Histologic analysis of the mutant embryos shows that the most severely affected structures are the digestive organs. Notably, the intestinal defect suggests arrest of development at a discrete stage. Normally, the intestine on day 4 displays differentiated organotypic cytoarchitecture: simple, columnar epithelial cells with a marked apical-basal polarity line the intestinal lumen. In contrast, the mutant intestinal epithelial cells are squamous to cuboidal, unpolaried, pleiomorphic, and fewer in number, similar to a 48-60 hpf embryo (FIGS. 1G and 1H). We were interested to know which developmental milestones the mutant epithelial cells had achieved, and chose to examine three dynamic parameters to this end: cell polarity, gene expression, and cell proliferation.

The cell polarity markers we examined were zo-1, a tight-junction protein localized to the apical domain; Na/K ATPase, which is localized to the basolateral domain; and actin, which becomes localized to the apical cytoplasm. The results of this analysis show that protein sorting and membrane polarity is established in the mutant cells, but actin fails to localize to the apical cytoplasm. Thus, the npo gene is required at this juncture in cell polarization.

We also examined expression patterns of genes known to be expressed during various stages of gut development. FoxA2 (formerly forkhead 2) is expressed in the endoderm from the mid-blastula stage through organogenesis, and we found no discernible difference in mRNA levels between the npo mutant and wild type embryos at 24 and 38 hpf Sonic hedgehog (shh) mRNA expression was also unaffected at 24 hpf, as was patched-1 and -2, indicating that shh signaling is intact. GATA-5 is expressed strongly in the intestine on day 4; its expression is retained in the mutant, but at a lower level, perhaps due to the fact that fewer intestinal cells are present. We tested terminal differentiation markers for the three cell types that are known to arise in the zebrafish intestine: in situ hybridization to intestinal fatty acid binding protein (IFABP) mRNA for enterocytes, in situ hybridization to chromogranin mRNA for enteroendocrine cells, and PAS cytological staining for goblet cells. This analysis showed that there was virtually no expression of these markers, indicating failure of intestinal cell terminal differentiation.

By bulked-segregant analysis, we mapped the npo mutation to linkage group 6. Genetic fine mapping placed the npo interval between microsatellite markers z8532 and z4950. Using z8532-specific primers, we initiated a chromosome walk and covered the genetic interval with two overlapping BACs, b37 and b90. We determined the complete DNA sequences of the BACs, and used internal microsatellite markers to narrow further the genetic interval. Two genes are contained within this interval, one encoding EphA2 (epithelial cell kinase) and the other encoding a putative protein with multiple RNA recognition motifs (RRMs). Complete sequencing of both cDNAs isolated from the homozygous mutant and wild type embryo revealed a tyrosine to stop codon change (TAT to TAA) in the RRM protein at amino acid 221 of the predicted 970 amino acid RRM protein. We found no mutations in the EphA2 gene that would result in any codon changes. In vitro translation of the full-length RRM protein-encoded cDNA from mutant and wild type embryos produced proteins of the expected sizes, 30 kDa and 110 kDa, respectively. Bac b37 contains the full genomic sequence encoding the RRM protein, but only the C-terminal portion of the EphA2 gene. Injection of b37 resulted in rescue of IFABP expression in over 90% of genotypically confirmed mutant embryos (23/25). Based on these data, we concluded that the RRM protein corresponds to the npo gene. Thus, the npo gene product likely binds RNA as part of its function.

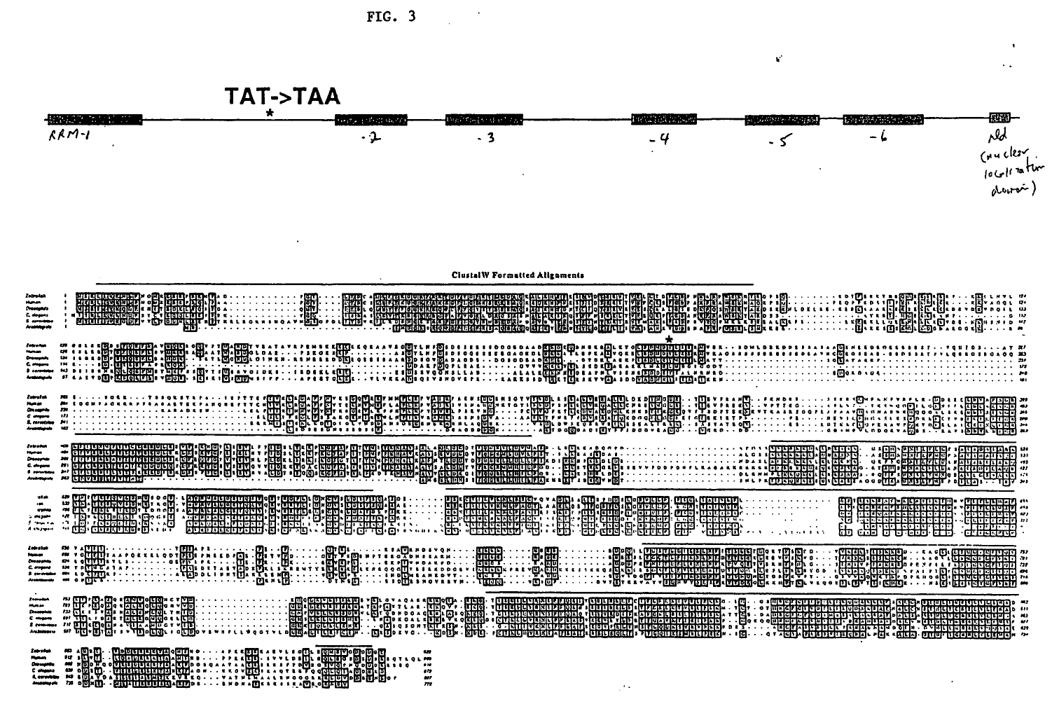

The npo protein contains six RRM domains, which are highly conserved in all eukaryotic organisms (FIG. 3). This multiple sequence analysis shows that the zebrafish protein shares 59% identity with the human ortholog, and about 30-40% identity relative to Drosophila, C. elegans, S. cerevisiae, and Arabidopsis. Using ³⁵S-labeled npo protein generated by in vitro translation, we demonstrated RNA binding preferentially to guanosine. The only description of gene function of any npo homolog is from the yeast gene deletion project. The homologous yeast gene, MRD1, is essential and its transcript is downregulated on diauxic shift. The human gene maps to chromosome 12q24.13 (termed DKFZp586F1023) and, prior to the present invention, had no known disease association.

The zebrafish npo mRNA is expressed maternally, and is non-specifically expressed in the early embryo until about 24 hpf, when expression is localized to the head and endoderm. The brain and eye mRNA expression diminishes by 60 hpf, but persists in the digestive organs until about 96 hpf, when it becomes barely detectable. Ectopic overexpression of the npo protein in zebrafish embryos is lethal, causing gastrulation defects.

### OTHER EMBODIMENTS

All publications and patent applications mentioned in this specification are herein incorporated by reference to the same extent as if each independent publication or patent application was specifically and individually indicated to be incorporated by reference.

While the invention has been described in connection with specific embodiments thereof, it is to be understood that it is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the appended claims.

What is claimed is:

1. A method of determining whether a test subject has, or is at risk of developing, a disease or condition related to a nil per os protein, said method comprising analyzing a nucleic acid molecule of a sample from the test subject to determine whether the test subject has a mutation in a gene encoding said protein, wherein the presence of a mutation indicates that said test subject has, or is at risk of developing, a disease or condition related to a nil per os protein.

2. The method of claim 1, wherein said test subject is a human.

3. The method of claim 1, wherein said disease or condition is a disease or condition of the digestive system or cancer.

4. The method of claim 3, wherein said disease or condition is of the intestine, the liver, the bile duct, the pancreas, the stomach, the gall bladder, or the esophagus.

5. A method for identifying a compound that can be used to treat or to prevent a disease or condition of the digestive system or cancer, said method comprising contacting an organism comprising a mutation in a gene encoding a nil per os protein and having a phenotype characteristic of a disease or condition of the digestive system or cancer with said compound, and determining the effect of said compound on said phenotype, wherein detection of an improvement in said phenotype indicates the identification of a compound that can be used to treat or to prevent a disease or condition of the digestive system or cancer.

6. The method of claim 5, wherein said disease or condition of the digestive system is of the intestine, the liver, the bile duct, the pancreas, the stomach, the gall bladder, or the esophagus.

7. The method of claim 5, wherein said organism is a zebrafish.

8. The method of claim 5, wherein said mutation in the gene encoding the nil per os protein is the nil per os mutation.

9. A method of treating or preventing a disease or condition of the digestive system or cancer in a patient, said method comprising administering to said patient a compound identified using the method of claim 5.

10. The method of claim 9, wherein said disease or condition of the digestive system is digestive organ failure.

11. The method of claim 9, wherein said patient has a mutation in a gene encoding a nil per os protein.

12. A method of treating or preventing a disease or condition of the digestive system in a patient, said method comprising administering to said patient a functional nil per os protein or an expression vector comprising a nucleic acid molecule encoding said protein.

13. A method of treating or preventing cancer in a patient, said method comprising administering to said patient a compound or molecule that inhibits the activity or expression of nil per os in said patient.

14. A substantially pure nil per os polypeptide.

15. The polypeptide of claim 14, wherein said polypeptide is a zebrafish polypeptide or a human polypeptide.

16. The polypeptide of claim 14, wherein said polypeptide comprises an amino acid sequence that is substantially identical to the amino acid sequence of SEQ ID NO:3 or SEQ ID NO:5 or comprises the amino acid sequence of SEQ ID NO:3 or SEQ ID NO:5.

17. A substantially pure nucleic acid molecule comprising a sequence encoding a nil per os polypeptide.

18. The nucleic acid molecule of claim 17, wherein said nucleic acid molecule encodes a zebrafish polypeptide or a human polypeptide.

19. The nucleic acid molecule of claim 17, wherein said nucleic acid molecule encodes a polypeptide that comprises an amino sequence that is substantially identical to the amino acid sequence of SEQ ID NO:3 or SEQ ID NO:5, or comprises the amino acid sequence of SEQ ID NO:3 or SEQ ID NO:5.

20. The nucleic acid molecule of claim 17, wherein said nucleic acid molecule is DNA.

21. A vector comprising the nucleic acid molecule of claim 17.

22. A cell comprising the vector of claim 21.

23. A non-human transgenic animal comprising the nucleic acid molecule of claim 17.

24. The non-human transgenic animal of claim 23, wherein said animal is a zebrafish.

25. A non-human animal having a knockout mutation in one or both alleles encoding a nil per os polypeptide.

26. A cell from the non-human knockout animal of claim 25.

27. A non-human transgenic animal comprising a nucleic acid molecule encoding a mutant nil per os polypeptide.

28. The non-human transgenic animal of claim 27, wherein the non-human transgenic animal is a zebrafish.

29. The non-human transgenic animal of claim 28, wherein the non-human transgenic animal comprises the nil per os mutation.

30. An antibody that specifically binds to a nil per os polypeptide.

31. A method of identifying a stem cell of the gastrointestinal tract, said method comprising analyzing a pool of candidate cells for expression of nil per os.

32. The method of claim 31, further comprising separating cells that express nil per os from said pool of candidate cells.


SEQUENCE LISTING (5 sequences)

SEQ ID NO: 1 - DNA, 23698 bp, Danio rerio
  misc_feature 6485: n = A,T,C or G
tggaattatgaacacattagtttacaaaaattagctttatttacgagagtgaatctataaaacaacaaaatcgcctcatatttatgacttttttttggagatcccccctcctaatctttaatatattttatgcttgaaagccaggacatacaaaataccaagcagcattaaataaatcttaataagtgttgttttaagtaaagtagcttcccttttaaggaaatatttcgcttatgtccacaagtgggagccataatcaagtgtctttggctacgcgctttacgtcacacagtaaagatggcgacgcactaaagggctccatgctttattgttgataaaaccatacattgttattttacaggagaaactctcacttggctcagaaaaatgtcaaggttaatagtcaaaaatctcccgaatggggtaagtattcataataatattagtatatgatagaactctgctgtcaagtatgcatgctttacgttatacgaagaagtgttcatctaacgttacagacacgtttatttgttgtatttactcacaaaaagtaacacaaaacgactgtggtttagtttgacgtgctgttgctgagctacagtggctactaaaagcagaataatatctaacagttacattacctattgacataaagggaaaagttggttttatatttggatatcccgacattctccttaataatataattttattaaagtattctttgcgaattgtaaatagggaaatcaaattagcagcaaatgactatcaaagtacagcattgttcacagtgaattagatagtgttaatgttgtttagaagtcatacttgcatgctaattccgatcgaatattcaaggtaacatggtaaaaattaagacgttagagaacaatatgacttcttaatgaacgaatgtgcgaatatgaaaccaactttttttacctcagccatttagactccttttgaaaatttgagtaaaaccgtaacgttagtaactatgttttttggcacactattaattttataacctcagtattacttcctatactaacgttatttttaaattatgattcgtgtagcaaaatcatttgccagttgtggtcatacatagaggtaaagttggttttatatttgcacatttgttcatttagaagtcatattgttcaccaggttactttgaattttcgattggaattagcatgcaagtgtggcttaaaaacaacattaacaatgtttatttgactgtgaacgatgttgtactttgatagttgttggctgctaaaatgatttcactatttagagtttgcaaataatacttttataaaatgatattattaaggagaaagtctgaatgtgcaaatataaaactaactttacctcaattggttatacgtggtttaatacgtagagtaatattgttttttgtttaattttcctaagagacagcaacagtgtaacattgtaccattactgtaccttcttataatcatcactgatatcattaaaaatagtttattttattccattaaaccctaaaatcaggcagtttgtgttctcaaactgtatgtgtgtgcaatgcatgtcatttgatctgctgataagcattttattcatgttgtgtctccagatgaaggaagagcgcttccgtaagatgtttgcagattttgggacacttacagattgtgcactcaaattcaccaaggatggaaaattccgcaaatttggatttgtgggtttcaagacagaagaagatgcacagaaagctttgaaacatttcaacaagagctttgtggacacatctagggttactgtgagtgacaagatcatggagcgtttttttaagcatttcaaaaaattataaaactatagtacagattttgtcccccattgttaacttgtacttttatttgtttttttttttctgagaaaaataattaaattttgtgcattatcatgatttgaaaaagacacacacaacctctccaaatactctttaattattattactttgtcactatccttcaatccactaggttttacatattatttttcaggaagaagtttttattaatttaaaatataaaacaaattagcatttctgctttttcttttatatatttaagtaagtacagtttggaataagaaaatgttgtttaagttgtcagttgtaagtcgttttggacaaaagcatctgctaaatgaaaatgttacataaatacatatgacattatttcacccttcatttatctaaattttacacacacatgaaaaatgggatgatcctaatttggtgtgttaatttgatacattttatatacgtttatataaatacattatttttgctaaaatgctttttaagttgacattaaaaataaatcatttatgaattaaaatagcaacccagatggtttaaatgaacattataaaatgccatttagatttttttatcatctgtccaaaaaaaggtggaattgtgcacagattttggcgatccaaacaaagccagaccttggagcaaacacacacgccaaccttcaaagaaagatactgaagaaaaaaaaacacatgagcaaggagaaaaagaaaaaaaggttggttaactaatttctgcctttttctcagcacttacttccaataaatctgattgcaaactataaagtaagtgcattgaaagatctgatgtttgccatttattttctagtagtttataactgaatttattgtaaaatatgcattacaaataaatcattctgaatttatgctcttgctccacagaagccgaagaagattttaaatgttcttggagatgtaagtttgcttttttgttttgtgtcagcacagttcatacaaaatcgaggaacttgctgacattctataaaatgttgtttgtaattctcagcttgaaaaagacgagagcttccaggagtttctggcagtgcaccagaaacgtggacaggttcccacatgggccaatgacacagtggaagcgactgctgttagacctgaaatggagaagaagaaagagaagaagcagaaagcggctgttgaagatgattacctgaactttgactctgatgaatcggaggaatcaagtgatgacggtgaggatgctgcagatgaagaggacaaacaaggtgtggtatatgtacaatatttaatttaacaaattttagcactgattacgatgtgcattaactctttgactgcccctctaccaaatagttggccatgtttttactgttttaaataatgtctgttaaagtcatttaaagaatgattgttttaaataatggcttacacgcattgttggtttacaaaaaaaacaaacaaacattctgttgcactattgcactatattactatacttgattttgattttattgtaaaaaacaaacaaacaaacaaacaagaaaacatgttaaatgagaatctgattgaggtgatttagtattcaaaaatgttttattttgaatatttaaaaaaaataaattagtcttacacttcatattttctgatttttcatagtatgtgtattgactgtgttacttttaccataaaccaacatatcaaccatttggtagtaggcatgcaatgattaaccgatttccctattaatagcaattaattcgtcacagttaaataatcataaaggcttttcaacaccgaatttttacacaaatgaaaatgcatcaactaaacagagttatgccaactgtgcactagtttaaagttccaaaattgtacactgaggctaaaacccacgcacacaggattaactatggctgaggccgttaactaagggcctttcacaaatcgggtcttatgtgcgcgcaagttcgttattttcagtggatgtacgcggcttacatgtgctaattggcaggccacaacacgctcgtgcacgctttaaaggtgcacctagttaaaagaatgcaatgaccgcaccttgacaagaactggaacaatcagcttcatactttgtattgaatatacacatttctactacaaagagagaaaaaaaaataaaagtaccaaacaattttgtaatatagatcaaacttgcctttcagacagaggttcagcagcatttgactacatttgttctctttaaaagagaaagatttacattagacaaattcattttatttatttttatttatttatgcattgttctttcatttattttcagtgtaaaattattacttctgttcaaatgccaaaacctttttttccacttatttttaagtgcaaagagaaatggactactgtttgttttttatttttatttttgtgctgtattaattggttactgagcagcaataaacaacagtttaaatataaggagttttcatgtgatttactagaagtgttttgaaattaaaactgcataataattgtgataactgtgattatttctcaggctgtaattgtgctaccaaaatccataattgttgcatccctatttggtagaggctgatattttagaaattatggggtttgttggaaaaaaatgcattcagtgtgttaactagcgacattaggagttaagaaatgcaattcaaacagtttttttctcttggtggctagtttaagggttaatcatgacattgtcgatatcgcaatgtgcaaatatgcagtagttacatcgcagaagctgcaatgttgagttgtgattataattaaccaggaaaaagttcagaacacaagcaaagcttaaccctaattgagtgaacaaagtgtgtgcaaggtttcaagttaatttaaaccatttgggtacaagaaattagaaaatatgtagctttaacatagattaattgtattttttttattacaatgtagattatacagtgttgatatttcacatccgattacactgttatttgacattttatcttaaaatgggtgggtgggtgattggtgcagatctgcagaggggatgcgcgcgtactgaagagcggtgttatcgcgcgcgtactgatcagctgtgttgtcgcgcgcgtactcaagagcggtgttgtcgcgcgcctactgaagggcgggaccgaaggtgtgccgcgtcgcgggggcacttttgatcattttggaagggcactttctatccaagactaaaaagggcatgtgcactgcacaggttgagccctatgtgtgcacgtgcctggtgctctgaatcgggctcaggcccagtaggaagaggtgttactgagcgcggttcaattgggggtttggcgcgatacgcttgtgtgtgagtgcaaaacgaaactaaaagtgagacgtgactttaagggtgctgtttcatatggattaattgatcattcttactgttcaatgaacgcaaactgtcgtagtttattaaagatgcaaacccctcactgcacgacagctgtgcaccttcagcaaacctcttaattcctgcagcacgaggactttgattgtttctgagcgtcaaaaatggttgatctgttcggcgaaatatttgtctgcgtgtcactgcatatcaaacgactaaaacgatataactaaagaaatctccacagtgctgagcgaaagagtttactgaacagtgcatcatcgatgatgttagcgtgcccaggcccgagtgtaatgtgagtgtgggccgtcgggggagatgggaggggggacaagcgtgctttggcccagttcaaggcaactgtacatagtgtgagtacgccctaataagcaacctcctatgctctatgctacaaatataaaatcatgaattcttagcaaacataaagttctttaaataatcttttaaaattacatgaatattgcagtaaatattgcagaaaaactaaatattgcattgtcagtttcgtccaatatcgtgtagctgtagttttgattcccttatcctttatcacaaagttttagagatggattttagtgaagttcatgtattgataataggatcaccttcattaatttaccctgagaaaaactactgtgtaaaacctacaacaacacatgtcaagatttttaatggtgttctgataaattcaagtgtgaaaatgaagtaatctttgacagtttaggcctgatgaattgcatcattgtcattctggagtattctcgttgaaaacatttttaccacattcactattatatgataatgtatatactatatactaccataatacttaatccagaattgatattttagaaattaaaagctacacacaggcatcatttagtgcaatgtaaagatgcagcatgcaatatagacaggctagcaggctacaatttgaatacaaaatattgacaaatatggggttggcagattgacaacaccaaacaggattgagcatagctgacaatggactttaggcgcaaatacatatccacaatgttctttttttccagtagatctctcatcactttttaaattgatggcagttgggtcaacatcataaattattctgctgacaaaataaccatgtggctgttacctcagcccagttgtttanttagttagaaggccatccaactggtcctttccctgtttaaaatagcgttttttgatgcattttatttaattttttctttaatgtgtgcattatttcaccagatgcaacatgtaaagacactgtcacctcagcttaatgtctgaaggaaactagcattcatatgttacagtaaaataattggctttagcattgttttttcgagaaagtaggtcaaattagcatatttatagataattttatcaataaagttacttacacatcatattctaaagaattgtttgtctaaatctgttttctcaccagataacgagaaggaggccttgaagactggtctgtctgatatggactatctccgctccaaaatggtggagaaatcagacatgctggatgagaaagatgacgacagcagtgcgagtgctgctgatgaaaatgaggaagatgaggggaaagaggaggaagagtccacagtccagcacgcagacagtgcatatgagagtggagagaagacaagcagccagaaaagcactaggccagcagtgagtgtaataacttttagtaagcaaatatggatttgtctctgttagagagtaactctgtgctcttcatgcattttattacagattgagccaaccacagagttcacagtcaagctacgaggtgctccatttaatgtcaaagaggtgagatttttgttattgggtgatattgaatgtgtaaaactttttatttttattttttacagcatccttagttaatgttacagcaaataaaccaaactattacattgatattaactgtttaactttttatgaagtgctgtctttagcagtgcatcaatatgaacattttatatctattaacattgacctatttttatgaatgataaaaaaaaatacataccaatttttttgtgataatgtggctgccgattgtacatccctagctatcttaactgatctgttattataatgatttatttttctaatttaaacagtttattgtagcattcatgtattacatttttacatatataattaaatttatgttaatgttacgttaatgtttttgcaactgtttataattacaaatcagttcttaaactgaccacaaatgtttctagagtaaatattaataagtgtaaaatgtttgtttctatcctacttttacacacttcgatataaatttgtgatgctactatattttatatttagtttaattaattattagaaatgtagtctgctgtttttttaaattcatgcgtgcaaaattttagtttgactgtttttaaaatagttaaaacagtttttaaatgtgtttttaaatgtgtgtgcgtgtttgtgtgtgttatgtcatatcatatacacttaccggctactttattaggtacaccttactagcacctggttcgtggccttcgtggcatagattcaagtaggtgctggaaatatttctcagtgattttagttcatactgacatgatagcatcaaatgctgcagatttgtgggctccacatccatgatgcgaatctcccattccaccacattccaaagctgcactattggattgactgtgaaggctgtttgagtacagtgaactcattgtcgtgttcaacaaaccagtctgagatcattgacacaccaggcaacgtttttccaatcttattttccaataataaagtgacctaataatgtggccggtgagtgtatgtgttgtgtatatgtccgtccctatgcacatctccttaacatttctgattttagttgtaatgcagtaatgtgcaccttatgtaaacaataattattgtgaaaatacaaataaacttaaattaaattgaatgtattttattttgaccacagcaacaagtgaaagagtttatgatgcctttgaaacccgttgccattcgattcgctaagaacagtgatggccgcaactcgggtaagagcatccgcttttttctgccggttgatgggttgatgatgtcaagctgctgcagattgattgatgttgtatttcctgcaataaggttacgtgtatgtggacctacgatcagaggcagaggtcgagagagccctgcgccttgacaaggactacatgggtgagcaccttttcactctttctgacaagttcactacagtgatgacctttctttgcttctgacattttttacaggtttaatctaatttgaaaacaagtatggtacatgaaactagttcccgtttgttcattatctgtgtgaacccacaggtgggcgctccattgaggttttcagagccaacaactttaaaaacgacaggcgttctgcaaaaagaagcgagatggaaaagaattttgtgcgcgagctgaaagacgacgaggaagaggaggatgtcgcagaatctggacgacttttcattagaaacatgccctacacgtgcactgaggaggatctgaaagaattatttagcaaacacggtcagtcgcagacacatgataaatattgttataaacaacaagtggatgacacaaaatacaggtgtaaacagagtatgttgagcttgtcttccagtggtagtcaaaaacactttttgtctggattaattttgtaatgtagactcatgtcagggagtccgcaatgtcttaaaaggtcttacatttcaaaaactaaatcttaggctttgaaaagtaatggattcactgaaatattgtgttgtaggtctcaaataattcaataaagtcttaattttcatatgtccatgtgaatttatccgatcagtccaacacccaccaaatccccaagtaatagaacttttaacaaaagtttaattttaactctgtttaccaacatagtttaattatcttctctacaatagcatttgattaaaagctctccatgtatttatagctatacctggtgaggaaactgacctggagagactgttgagcatatgtttagtttagtacaatttaaaaacttttttttaaagaaatgttatgtaaaaaaagtgcatgtagactagatataactagagtttgcttgttttgacacattatttaaagtatgtggctaagaaataactaaattagatttatatttagttttttgatgaggcaagtaaaaagattttccatttgtccaaccaggccataagaaaaaaaatgccttgtgtttagccctctataaaggtctttgcacactgaagtcctaaatgttcgtgtgcgtttttttttttttttaaatcatatgcaaaaaaatcatcatgtaaacaaaccttgcacactgactccgatgtgcagctcattatcaaaaatctgcgctggattatcccgaagtttatttcaataaatcagtggaatttaatacattgcttgtgatactttacacattcacgtacataaacaaatcctgaaaagagactgcatattaaatgacattataatagatggtggctgcgctgacgtatcgaagcagcagaccgaaagtggaccgtgctttctattataatgtctatgggcagtacgtcaaatgtgtatgaagacacacacacacacacacgcacacacacacgcacacacacacacacacacaaaccaagcagcagcaggggagaggtgcgccagccactaaatccaggataaaggggttctcagctgtccgccacattctgtctttatttcatgctcttaatgtttgtcataacgttgtctgtagctcagatgatggcattctgtaattagcttttcacttgtaaggtgagtctaaactgttaaaactgtcaaaagctatttttttaatgacagacgaaagtttcggaggcagtgtgtcaggtaattgacacaacgtgaggtcgaatatattttttagagtatgaaaattacagatgaaaatttcagattcagtgtgcaataatacccttatgtcagaaactttattcataatggtcttgaaaagtcttaaatttgacttgatgaaacctgcagaagctctaaatgtggtctaatgcaatgcaacagccactgattgttcactagtagatggaatttaaacgttgctgttaaaatggtaatttcgatttgatggcaaaaattcttaaaagctaatgttctcttacagtcctggtcctataaatagtagttttgcatttattagagcagaaatcaaagcttcaaactaatctttgttgtctcatcaggtatttatgtatgatctctgctgacttattctgtgatattagaatgatgagaaattgacaatatatctatagactcttaaagaattcagttaagggattgaaagtggacaaaagattaaaaataaaacatcagttgtaagtaggaaacggttgtaaataccaatgacatactaagcactaatacctagaaaattcattttatcttctcccaaattctgttttacagattttttcttggtttcttttttaaatgtacatttttatgctttaaataaaactttttcttttttaattttggcttttgttttagcaatactttacacaaccaaaattctgaaaaaattctgaagttacccaacgtctgtgagatcacgttatgcagtaacattacatttagtcctgtaaatgaatcaaattttttttacgtttttatcaattaattatgcatgtgttttctttaatgcataccttaatttcttggtctatttttaaaaaacaattattttcttgtgtcccccatcaggtcctctatctgaggtgcttttccccatagacagtctgactaagaagcctaaaggctttgcgtttgtcacatacatgataccagagaatgcagtatcagccctggctcagctggatggacacacattccaggtactgatctgttcttttgacctcagctttgactattggggctgtctttgttttcttacctctgtttggacactatagggtcgtgttctgcacgtcatggcttcaaggctgaagaaggaaaaggccgatcaggggcctgatgctcccggcagctcctcatataagagaaagaaagatgccaaagataaggcagccagcggcaggtcaggatctgtctggagctgaaaaaacttgcactttattttaaggtgtctttgttacagtgtcactatacattatacaagtactgaagagttaaaattgactgcaggtacttgctgtatatatggttagagtcagtgtagggtgcaatgagttacaaagaaattaaattacatgtttagtgaaagtaattaatggattacttttattttggaggtcatttaattacagttacttataatatacttgtcttatatactgttaataaatttagcagttttacattggtgatagtgttatactgggttattgtctgcttttatttttggtgttggattaatatttgctagcagggaaaatgttttattgcataacaattagtttatcaggctttttggctaaaaaagcgaaatcactgtttaatttgctattattagaatatttttaaatttcccctatccttctgtgcatgaactaagggtttattgttgttcttatagtaagtacatggaatgtgttagaaacaaagaccacaatgttaccagttgtcttctctttattgcctttttaattcagctctcataactggaacacgctgtttttgggtacgagtgcagtggccgatgccatcgctgagaaatacaacacaaccaagagccaagtgttggatcacgtgagtctttgagtcgtatgaatgctctttattttgttttggagatgataaacaatggacttgcttctgtttgtgtgcaggagtctgatggcagtctggctgtcaggatggctcttggagagacgcagattgtacaagaaaccagacagtttctcctggacaatggagtttctctggactcgttcagtcagggtatagtgtttcttttactcatacactattcattagtctgggttcagttcaatttttgttttaattactttcaacatgatacctcactcctaaaatgaaaatcctgtcatcatttgattcgtttcagagttttggggttctgttgaacacaaaagaagatatattgaaacctgctgcgattttcttacatagtatttgtttttgtttttctgtgtattccaaaggttgcttgtttccaacattcatcaatatatctccttttttaaaacaaacaaacaaacaaacaaacaaacaaacaaacaaacaaacaaacaaacaaacaaacaaacaaagacaaaaacaacctcacactggtatggaacaaatcgagggtgagaaaatgataacaattttcattttcgggtgaactattccttcaaacttatcaagaataccagtaaaacattgatgtttttccacgtacagttgtttttgcaaacacattagtttatatgacagctttcagtattggtaataataacaaatgtttcttgtgcatcaaaccaactcattacaataattctgaattttagccctcgttcagtcaggtatagtgtgtttgatttacccatacactgttcaagttcactgttcagacactttacagaatctgcattcagtacaatttttattttaattaattactttcaactatggggtccttaaagggattttacaccccaaactgaaaagtcctgtcatcattcgtagcctttcaggcattttcactgaaggagatatactgaaacctgtaaccattttcttccataatatttctttttttgctactatgaaagtcaatgtttacctgtttccaacattcatcaatatatctcctttgaaaaaaacccaaaaaaaaaaaacccaaagaaacaaaaaaagaaaaaaaaactcaaactggtatggaacaaattgaaattgatgacagaattttcatttttgggtgaacttcctttaatctcatcaagaatattaaataactttcaacatagggtacttaaaaggattcttcaaccaaaatgaaacccctgttatcatttgatttgtttcaaagttttggggttcttttgaacacaaaaaggagatatactgaaagctgtaaccattttcctcccaagtatttgttttttttcttctgtagatgtcaatggttgcttctttccaacattcattaatatatctccatttttaaaataaaatataacaaacaaacaagcaaacaaaaaactctcaaacaggtatgaaacaaattaagagtgagaaagcaatgacagaattttcgtttatgggtgaactatccctttaaactgatcaaaaataccagtaaacattaatgttacaaaaaagactaaaatttcaaagaaactgtccagtttgatttttctgttcattaaataaccctataaaatgttaaatgatatttgcaaaagtgttagtttctactactgttttcaccattgataataataactaatgtttcttgagcatcaaaccagctcattacaatcatttctgaaagatgacaggatgctgaatacactgagtgagacattaaagctgaatattcagctttaaaatgttatttttatatacattgtgcattcttgattgaatcgctcgcattattctttaacatctattgaaataacaacgcatcgctgaatcgctcgcattattctttaacatctattgaaataaaaacgtgataatgattcataataagggcacatttaaaaaaaaaaaagcttgatagaacaacatcagtgtccataaactgactcctcttcatcatcatcttcagacaatgctgaccaaaagttatggaaatcatgtcgatcaacagtgtggtcatcatttactgcattgacaaatgtgctagcacaatgtcaaggtgtatctgaggctgtaactttgcaatacttaaacatattgaaataacaatgtgataatgattcataatgattcatagtaaggacacatttaaaaaaaaaaaaaagcttgatagaacaaacatcagtggccaaatccatcttttctgtttgtatccacatggttaactcatctatatgtttaaacgtgtgtggtttccaggcgtcgggtgagcgcagtaaatgtgttatcctcgtaaagaacttgccgtcaggagtgcaggttgcagatctggaggctctgttctcgccccatgggtctttgggtagagttctgctgcccccttctggccttacagcgatagtggagttcctggagcccacagaagccaaacgtgctttcatgaaacttgcatacacaaaggtcagtgggcacttgtttggaatggccatttcagctaatgtttaggctatgttttattggggctttagctagtttaatcaagctttaatgacagcatatgatgttatatgtttgtatttctctttctagtttcaacatgtccctttgtatctggaatgggctcctgtcgctgtctttacaactccctcagcacctagaccaggtaatactttaattctgattcagccatctgctgtaattaaacaatcaattcaatgttaggtgactttgatgcgttttcatttgttgtccctttttttctcattaataataagtaataataataaaactattctagagtacatcccaatctgtgtttctatgatttctttttcttatatttgttttttgttgtcccaatctgtgtttctgtgttagttctttgtgttattctctgattctgtttttcctcccagagcctcaaaccaaagagaaatctgctgtgaaaaacgattcagtccaaaatgaagaagaagaggaggaagaggaagaagatgaccagattttacctggctcgactctcttcattaagaacctgaactttatcacatcagaagaaacattacagaaggtaaactaaaacacacacacacacacacacacacacacacacacaaacacacacaagcatatgcttgagcagcaaagcggttgtggtttagggatttgcagtggaaacctgagaggccttcttgttttgctgatcacaggagactcatttaagatgcatattagttctaggtgtaaacatgtgttgtgtgatcagatcacccgaaacggatgttcataacaggtgtaaacatggcccttgtctcaccatatggctttcattgcattcttttgacagacgttttctaaatgcggcgtggtgaaaagctgcacgatatcaaagaaaagagataaagcaggtttgttttctccgaaatgcatatttactgtattgatattcatctgtgcgcttgttagttttctcatttctctgtctcgtttcagtcgctgtgtgaatctcaccctgatctctttgtgtgtgtgtgtgtgtgtgtgtgtgtgtgtcatgcatctcaggtaaattgttatcgatgggttacggctttgtgcagtacaaaactccagaggcggcacagaaagccatgagacagctgcaggtaaatgaccatctgctgtgctttcatcgctgtgtttatgtgctcttattatgcgcaaatattcaagtgatgaagtaaattcaggcatgttttttcctcgtttcattcatttttcttgcagcactgcacagttgatgagcaccagcttgaggtgaagatatcagagagagaagtcaagtaagtcttttgtgtactccattttcaagtgcggtgtagatttgttaaagctcgtgaagacttctattctcgctaggcaatttgcagggtactaggttagatcatttttttgctatcagaactgcctcagttgtccataataatatgatttaaatagtaaaagcatttgtgtgccatggacctgctagtttgaagtgaaggtgtgttaaaggaatagttcaactaactaactagttagttttaactagtttcaaacatttatgattttctttgttctgttaaacacaaaataatatattttgaagaaactttcctttgtagccacatgtaaccattcaatttaattcatctttatttctattgtgcgtttacaatgtagattgtgtcaaagcagcttagttgcagttagttctggtaaattgaaaccgtgtcagtccagtttcagtccaagttgaagttcagtttagttcagttcagtgtggtttttcactgctgaaagtccaaacactgaagagcaaatccatcgatgcgcagctccacaagtcccaaaccaagcaagccagtggcgaggagtacacttcaccagttgacaaaagtgaaggaaaaaaaaccctcgagagaaacaagactcaattgggcatgaccatttttcctctggccaaacttcttgtggaaagctgcagtctaggcggcagaggctatagaacgctggacgtctatagtggagacttccattggcttccatagaatttgtttttcctcctagggaagtcaatgcttacaggtcttcaactttcctcaaaatatctttttttctgttaaacagaagaaactaactcataaaggtttaaaaccactttgatggtgcttaaataatgattacatttccttgctgtccctttgaggtgcattaatggtggattgcaaatttgatgcagctgcactgcaattttaccttccgagcccaaacatgcctatgtcaacaaagatgctactattctgttagaaagagaagagctctcgctcagtacaatggagattgctttagttagtatttttgtattatttttagtctattttttattttaaatttagtctattttagtggtttaggttgcagtgacacacagtcgaatatttagctgaacagagccaccactttggtgcctcaattttaatgaaaatcatcgtgttgcacattatgaggtttgctgaaggtccagctgacatgcaccttggtgtttgcacatttaataaactgtgacagttcgcttacattaaaaagtaagaattattaataaatctatgtcaaacagtccctttaaactgactccgtgtcttcagttttgggctcaggcgagctttgcattcacactacaagcgtattgtagtagacaaactgaaccgcactcctcttccaaccaggccagggtcggctaactgaaccggcgtgattcggagcactcacacttgtcaaatgaactgggaaatggcggtcaaacgtgccctgggtcggttcgaatagcataatttgagtatactcttaaagggcgcgttagtaaaatgcacctatacacgtacatacatgttgcttattacttaaagggacatgcagcagcacataaacatctacactatctacaaaaaaatgataataattatatgtcctgccatgcgttcttcacctcagggggcttttgtggaatttggctgtttctgtagatggtctgctgcattttaacctcacgcatgagcacatttgttttcttgctagggaagcattctgtttttccgcttacaaagtcctctatgtaaatagcgaatccgcttggcgcaaccacaactgactcgtgaaaggaatgggaaactgattggtttactgtgcgctgcccaaaacttattaagacaataaggattgttctcatcctttaagtagcaagagttgattcggacatgctctaagtacaacagccatgtgtgctttagaccatgcgcttagattgataaagtaaagcccgttgttttgttcaagaaaccattttgggttgttttgagttttctgacatcgtcctgctacaattggccataacaagataatcaaggtatggacattgtgaggaacaatactctcaaacttggcacaaacctgacccattgatgcaaagcagcatggttccatgaaatttttttttttttaacatcaaattctgaccctatgatccaataacacagtagaggttaagactcatcagaccaggcaatctttcttttttgtcttatattgtttaatttttataaatagtaatagggatagtaaagtatgagtaagtttacatttttgggtcaactcttctttcgtagttgcccgtgacattcattaatgtcattaaaatgtggcaccggtcacatgtatgagtgaaaaacgtgttccttatgttgtttttgtaggtcaggtgtggcacaggccaagaggaaaaagcaaaccgccaggaaacagacgacctctaagatcttggtgcgaaacatccctttccaggccacagtcaaagagctgagagaactcttctggtaaacatcctttcacattttaatgtttcatgccatattattccaacaagtgtggaatttctagcatgtatatggtaccttaacaatattcgtatgcaacagcatttttaataccagagagaaaatattgcattagcatgaagtaaaattaaaatgttttcacgtcttcattttttttttagacgtgaaggtttgaatggtttgaatggttaagatgttcacttgcattgccaaaatttaaccattgtttgaaagttgtttgcatcgttgtttaaagcaacgtcaatgctaacctagagttgcaggaaaaaatattagattattagaaaattatttgaaatttgaattcttcaaatatttccagggtgatgtgtaacagagcaaagacattttcactattcctattatatatatttttattctggataattttacatttttagtttggctggaataaaagcggttttatttattttttcaaagttatttttaaggtcaatattatttatattttttaagagatgataattgtttgattggctacagaacaaaccactgttgtcattgactcacctagttaagcttggctagtcaaaataatgttagtttagcctttaatttgcactttagactgaataatattgtcttccaaaccactagatgaatatcatttaaggtcatcatggcaaaaacaataaattaggtttaaaatgaaaactccattaaacattacttaggaaataattgaaaatacaattacattacaatttcataatgaatttgccttttactctatacagtggtccctcaccataacgtggttcacttttcaccacctcgcagttttacagatttttttagtgcaatttgacatgctttttttttccaaagcattgtgttctgtttcctgattggctgtaaagcattgtcaatcaatcaatcttctccatgccgtgtcactgtacagtacagaatgcgttcagcttgccaaatttacatgaatctttgatcactagcagtgtgactctgaagtgctggactgtacgttttctttccaacaaatcccataatgtcgaaaaaacattctgcacagacaaaggcacctgtggcaacccccaaaaggtagaggaagatgcgagcatcacacaaaaagttggacttcttggtccaacttggtccacttggagtgtttaaagaagagagaaaaaacaggaaaatgttaattgtaaaagtaaagcggactaaattgaagatttcacctattgcagactatgtttaggatgtaactctcccaagattaacgagggaccactatatgcactgtataatatagattactgcgaacattactgaagacaagtgatctgtaataaacaaattatgaccaataacactaataaatagaacagaacaaatgtacagatgaaaacagcgctttgggttgttctatttttgtcttttgttgttaattaaaaataaaaacttttaaagtatggcatgtcatttactgtaacatgtttccctatctgttaaagcacaagaacatgtaaaggaaaaagagatgcacaaacagtgctcgagtgttgagtttttttacttcagggtgtctgcagggtcttaaagtattaaaatgtcttaaatctcaaaaacaaaattgtaggcctcaaaaacttaaatttactgaaattgtgttataggtcttaattgtttttgtaaacaggtcttaatttttctctgttcatgtatagctacacaatctggccaacacccatccaatcaccaacaatctatttcagtaaaactttaaacttttctttaagaatgtcatttttaaactctatttaccataatggtttaattattttccaataaaacaataacaataaaacaataacagttgtttaaaagtgaggaagctgaccttgtcagacttttgagcatttagtttattatagtttttaaaacttcaatcattcattcatttttttttccggcttaatccttttattaatctggggtcgccacagcagaatgaaccgccaacttatccagcacatgttttacgcggcggatgtccttccagcagcaacccatcactgggaacacccatacactctcattcatacacatacactacagacaatttagcttacccaattcacctgtaccgcatgtttttggactgtgggggaaaccggagcacccgaaggaaacccacgcaaccgcggggagaacctgaaaactacacacagaaaggtcgctggttcgagccttggctgggtcagttggcgtttttgtgtagagtttgcatgttctccctgcgttcgtgggtttcctctgggtgctccagttttccccacagtccaaagacatgcggtacaggtgaattgggtaagctaaattaattggctgtagtgtatgagtgtaaatgagagtgaataagagtgtttggatgttacccagagataggttgcgggctggaagggcatccactgcatataacatgtgcatgcataagttggctggttcattccgctgtggcgaccccagattattaaagggactaagccgaaaagaaaatgaatgaatgctctttaaaagctgcagaaaccctgtactttgtttttctgcatttctaaactgataaaatacttatattgatgcataactactgcatattgccaatatttgctaaaaagtactattgtaatttcatggttttgtactctaatagaagtcgaggtgttgtagatgatgttggattcatgtctttccctcagtacgtttggagagctgaagacagtccgcctgccaaagaaagggattggtggatcccaccgtggttttggcttcattgacttcctcacgaaacaggatgccaaggtgtgtgcagctgtctctgtttctttctgttgctcttattttttacggtcaaacagctgattgtctttgattcggtgtagtgtctaacaggatctttgtatgtgtgacagaaagcgttctcagcactatgccacagcactcatctgtacggcaggaggctggtgctggagtgggcagatgctgaggagacggtagacgacctgcggaggaaaaccgcacaacactttcatggtaaaatctctgctttatacttcagccacagtcatggagagcagacgctgtgaaacagtttgatcagtcacattaaatgcaaacttaatttgacacactttcttagtagcctgacaaaacaagctgtttttggtagcatcacaggatgaaaaaaagatttgcgtttcgtttttatgacactatgaaatagaaagttttatggatttgtatggtcaatctctgttttcatttgagccccaaatacagtaaaaaactaaattattagccctcctgtgaaatttttaaatctattttttaaatatttaacaaattatgtttaacagagcaaagacattttcacagtatttcttatatatttttttcttctggggaaagaattatttctttttttttaattctaaataaataaaactaaatatcttttttgcattaatttttttttatacaattttaatgataatttttccttcaattgtacgttttgttgcatttatctaaacaaaataaataaaaatgtcataaaataaatgaaatagttttttttattctaattaaagcatgtaaatataatttttacattaaacttttaaaaaataaaattttaatgccgttcactacctgataaaagtcttgtcttcgatcccaattgtaagaacaacaaataataactcatttggaaaagtggcagaaggtcgagttttctttttttatttaattttttttccctaataaattattattatactattctattattatactttactattatactattaatactattatagtagtgtatgtggactctattatttagttctgctgctttagaaacataagcaaaaaacattaaccagaaacatctaaacagattgatctaaaatgctcagttgttatattctgtcaaattaaatgtccttttaaaagcaaagaatcttaattaaattaggagtaaattgaatgaatgtcaaatcctgtcaagtaattcagctcatcattagtttgtgccagatacaaaaagtgttgatttgtatttaatagcgcttaattgtttgaattgtttacttcgttttgtcaaactttttattttttggcgctactttgactgtcaaaactctgccaaaacacaagctactcctaatttgaagcgttttctctaacatcgaccgcaatcctagtcatttcccagtctgtaattaaattaaaacaaatctgtgcgcaaatctgtctccctcagatgctcctaagaagaagaggaaggcggaggtgttagagggaatcctggagcagatggaggtcggcgatggagacggcgagtgaatagcagccgtcagtcattcacctccagcatcaataagagagtaaatcatccagtgcaacttcatttatctctattatgactgcgtcttaaacagctgcttcacgggccctcttcaggaattcttccttttggccccagatgctcccaattagctttttattatatgctcatacatcatccctgttattgctgtcaatgcattaaacactgcgtctccctgcagacccgctccgactgaaggtgaactccaagactaagtccttttagaaaagcaaaagcccgaaggccacgcttgctttggctgtttttaatcgtcacagagggccgagacggttcatacactgccttgacacgcggatgacattgagaaatcgtatcagaaatgaaatgtggaaggggtttgattgttgtttgtacacacagagattgtgtattgtatttccagatgcttacataattatgtaaagtttttgtggtgtttaaagtgatggttcagcccaaagtgatgtgttattcactgtcaagtggtttcaaaacatttagtttcctgttttctgttgaacacaatagaagatattttgaagaatgctggaaatccattataaagaataaaaaatacttaagtggc

SEQ ID NO: 2 - DNA, 3274 bp, Danio rerio
  CDS (1)...(2778)
atgtcaaggttaatagtcaaaaatctcccgaatgggatgaaggaagagcgcttccgtaagatgtttgcagattttgggacacttacagattgtgcactcaaattcaccaaggatggaaaattccgcaaatttggatttgtgggtttcaagacagaagaagatgcacagaaagctttgaaacatttcaacaagagctttgtggacacatctagggttactgtggaattgtgcacagattttggcgatccaaacaaagccagaccttggagcaaacacacacgccaaccttcacagaaagatactgaagaaaaaaaaacacatgagcaaggagaaaaagaaaaaaagaagccgaagaagattttaaatgttcttggagatcttgaaaaagacgagagcttccaggagtttctggcagtgcaccagaaacgtggacaggttcccacatgggccaatgacactgtggaagcgactgctgttagacctgaaatggagaagaagaaagagaagaagcagaaagcggctgttgaagatgattacctgaactttgactctgatgaatcggaggaatcaagtgatgacggtgaggatgctgcagatgaagaggacaaacaagataacgagaaggaggccttgaagactggtctgtctgatatggactatctccgctccaaaatggtggagaaatcagacatgctggatgagaaagatgacgaaagcagtgcgagtgctgctgatgaaaatgaggaagatgagggggaagaggaggaagagtccacagtccagcacacagacagtgcatatgagagtggagagaagacaagcagccagaaaagcacaaggccagcaattgagccaaccacagagttcacagtcaagctacgaggtgctccattcaatgtcaaagagcaacaagtgaaagagtttatgatgcctttgaaacccgttgccattcgattcgctaagaacagtgacggccgcaactcgggttacgtgtatgtggacctacgatcagaggcagaggtcgagagagccctgcgccttgacaaggactacatgggagggcgctacattgaggttttcagagccaacaactttaaaaacgacaggcgttcttcaaaaagaagcgagatggaaaagaattttgtgcgcgagctgaaggacgacgaggaagaggaggatgtcgcagaatctggacgacttttcattagaaacatgccctacacgtgcactgaggaggatctgaaagaagtatttagcaaacacggtcctctatctgaggtgcttttccccatagacagtctgactaagaagcctaaaggctttgcatttgtcacatacatgataccagagaatgcagtatcagccctggctcagctggatggacaaacattccagggtcgcgttctgcacgtcatggcttcaaggctgaagaaggaaaaggccgatcaggggcctgatgctcccggcagctcctcatataagagaaagaaagatgccaaagataaggcagccagcggcagctctcataactggaacacgctgtttttgggtacgagtgcagtggccgatgccatcgctgagaaatacaacacaaccaagagccaagtgttggatcacgagtctgatggcagtctggctgtcaggatggctcttggagagacgcagattgtacaagaaaccagacagtttctcctggacaatggagtttctctggactcgttcagtcaggcgtcgggtgagcgcagtaaatgtgttatcctagtaaagaacttgccgtcaggagtgcaggttgcagatctggaggctctgttctcgccccatgggtctttgggtagagttctgctgcccccttctggccttacagcgatagtggagttcctggagcccacagaagccaaacgtgctttcatgaaacttgcatacacaaagtttcaacatgtccctttgtatctggaatgggctcctgtcgctgtctttacaactccctcagcacctagaccagagcctcaaaccaaagagaaatctgctgtgaaaaatgattcagtccaaaatgaagaagaagaggaggaagaggaagaagatgaccagattttacctggctcgactctcttcattaagaacctgaactttatcacatcagaagaaacattacagaagacgttttctaaatgcggcgtggtgaaaagctgcacgatatcaaagaaaagagataaagcaggtaaattgttatcgatgggttacggctttgtgcagtacaaaactccagaggcggcacagaaagccatgagacagctgcagcactgcacagttgatgagcaccagcttgaggtgaagatatcagagagagaagtcaagttaggtgtggcacaggccaagaggaaaaagcaaaccgccaggaaacagacgacctctaagatcttggtgcgaaacatccccttccaggccacagtcaaagagctgagagaactcttctgtacgtttggagagctgaagacagtccgcctgccaaagaaagggattggtggatcccaccgtggttttggcttcattgacttcctcacgaaacaggatgccaagaaagcgttctcagcactgtgccacagcactcatctgtacggcagaaggctggtgctggagtgggcagatgctgaggagacggtagacgacctgcggaggaaaaccgcacaacactttcatgatgctcctaagaagaagaggaaggcggaggtgttagagggaatcctggagcagatggaggtcggcgatggagacggcgagtgaatagcagccgtcagtcattcacctccagcatcaataagagaacccgctccgactgaaggtgaactccaagactaagtccttttagaaaagcaaaagcccgaaggccacgcttgctttggctgtttttaatcgtcacagagggccgagacggttcatacactgccttgacacgcggatgacattgagaaatcgtatcagaaatgaaatgtggaaggggtttgattgttgtttgtacacacagagattgtgtattgtatttccagatgcttacataattatgtaaagtttttgtggtgtttaaagtgatggttcagcccaaagtgatgtgttattcactgtcaagtggtttcaaaacatttatttcctgttttttgttgaacccaatagaagattttttgaagaatgctggaaatcccttataaagaataaaaaatacttaagtggcaaaaaaaaaaaaaaaaaaaaaaaaaaaaaaaccaacaaaaaaaaaaaa

SEQ ID NO: 3 - protein, 926 aa, Danio rerio
MSRLIVKNLPNGMKEERFRKMFADFGTLTDCALKFTKDGKFRKFGFVGFKTEEDAQKALKHFNKSFVDTSRVTVELCTDFGDPNKARPWSKHTRQPSQKDTEEKKTHEQGEKEKKKPKKILNVLGDLEKDESFQEFLAVHQKRGQVPTWANDTVEATAVRPEMEKKKEKKQKAAVEDDYLNFDSDESEESSDDGEDAADEEDKQDNEKEALKTGLSDMDYLRSKMVEKSDMLDEKDDESSASAADENEEDEGEEEEESTVQHTDSAYESGEKTSSQKSTRPAIEPTTEFTVKLRGAPFNVKEQQVKEFMMPLKPVAIRFAKNSDGRNSGYVYVDLRSEAEVERALRLDKDYMGGRYIEVFRANNFKNDRRSSKRSEMEKNFVRELKDDEEEEDVAESGRLFIRNMPYTCTEEDLKEVFSKHGPLSEVLFPIDSLTKKPKGFAFVTYMIPENAVSALAQLDGQTFQGRVLHVMASRLKKEKADQGPDAPGSSSYKRKKDAKDKAASGSSHNWNTLFLGTSAVADAIAEKYNTTKSQVLDHESDGSLAVRMALGETQIVQETRQFLLDNGVSLDSFSQASGERSKCVILVKNLPSGVQVADLEALFSPHGSLGRVLLPPSGLTAIVEFLEPTEAKRAFMKLAYTKFQHVPLYLEWAPVAVFTTPSAPRPEPQTKEKSAVKNDSVQNEEEEEEEEEDDQILPGSTLFIKNLNFITSEETLQKTFSKCGVVKSCTISKKRDKAGKLLSMGYGFVQYKTPEAAQKAMRQLQHCTVDEHQLEVKISEREVKLGVAQAKRKKQTARKQTTSKILVRNIPFQATVKELRELFCTFGELKTVRLPKKGIGGSHRGFGFIDFLTKQDAKKAFSALCHSTHLYGRRLVLEWADAEETVDDLRRKTAQHFHDAPKKKRKAEVLEGILEQMEVGDGDGE

SEQ ID NO: 4 - DNA, 3594 bp, Homo sapiens
  CDS (84)...(2963)
gcggcgcccagggcggtagcgtgaaacttggtggaagacgctgaccagtcgtgttggaatcaaaacagcggggaccctgcgccatgtcgcgactgatcgtgaagaatctcccgaatgggatgaaggaggagcgtttcaggcagctgtttgccgccttcggcacgctgacagactgcagcctgaagttcaccaaagatggcaagttccgcaagtttggttttattggcttcaagtccgaggaagaggcccagaaggcacagaagcatttcaacaagagcttcatcgacacatcccggatcacagtggagttctgcaagtcattcggggacccggccaaacccagagcctggagcaaacatgcccagaaaccaagccagcccaagcagcctccaaaagactctactactccagaaattaagaaagatgagaagaagaaaaaggtggcaggtcaactggagaagctgaaggaggatacagagttccaggagtttctgtcagttcatcagaggcgggcgcaggcagccacttgggcgaatgatggcctggatgctgagccctcgaaagggaagagcaagccggccagtgactacctgaacttcgactccgattctgggcaggagagtgaggaggagggagccggggaggacctggaagaagaggcaagcctcgaaccaaaggcagctgtgcagaaggagctgtcggacatggattacctgaaatccaagatggtgaaggctgggtcgtcctcttcctcggaggaagaggaaagtgaagatgaagccgtgcactgtgatgaagggagtgaggccgaggaagaggattcctccgccaccccagtcctgcaggaaagagacagcaggggtgcaggccaagagcaagggatgccagctgggaaaaagagaccaccggaggccagagccgagacagagaaaccagcaaaccagaaggaacccaccacctgccacaccgtgaagctgcggggagccccgttcaatgtcacagagaaaaatgttatggaattcctggcacccctgaaaccagtggccattcgaattgtgagaaacgctcatgggaataaaacaggatacatctttgtggatttcagcaatgaagaggaagtgaagcaagctctgaaatgcaaccgggagtacatgggtgggcgctacatcgaggtgttcagggaaaagaacgtccccaccaccaagggtgcaccaaagaataccaccaaatcctggcaaggccggatactcggggagaacgaagaggaggaggacctggccgaatccggaaggctctttgtacggaacctgccctacaccagcaccgaggaggatctggagaagctcttctccaaatacggtcccctgtctgagctccactaccccatcgacagcctgaccaagaaacccaagggttttgcattcatcaccttcatgttccctgagcacgctgtgaaggcctactcggaggtggacgggcaggtattccagggcaggatgctccacgtgttaccatctaccatcaagaaggaagccagcgaggatgccagtgccctgggatcgtcgtcctacaagaagaagaaggaggcccaggacaaagccaacagtgccagctctcacaactggaacacactattcatggggccgaatgccgtggccgatgccatcgcacagaagtacaacgccaccaaaagtcaagtgtttgaccacgagaccaagggcagcgtggccgtgcgcgtggctctgggggaaacccagctcgtccaggaagtgcggcgttttctcatagacaacggggtcagcctggattccttcagccaggctgcagcagagcgaagcaagactgtgattctggtcaagaacctcccggcaggcaccctggcggccgagctgcaggagaccttcggccgttttggcagcctgggccgcgtgctgctgccagagggcggaaccactgccatcgtggagttcctggagcccctggaggcccgcaaggccttcaggcatctggcctattccaagttccatcatgtccccctctatctggagtgggctccagttggcgtcttctccagcgcagccccacagaagaaaaagctccaagacacaccttcagaacccatggaaaaggacccagcagagccagaaacagtgcctgatggcgaaaccccagaagatgaaaatccaacagaggaaggagcagacaactcttcagcaaagatggaagaggaggaggaggaagaggaagaagaagaagagagcctcccaggatgtactctgtttattaagaatctcaattttgacacaacagaagagaagctgaaggaagtgttttcaaaagtggggacagtgaagagctgctccatctccaagaagaagaacaaagcaggagtgctcctttccatggggtttggatttgtggaatacaggaagccggagcaagcccagaaagctctcaagcagctccagggtcacgtcgtggacggccacaagctggaagtgaggatctcggaacgagccactaagccagccgtgacattggctcggaagaaacaagttcccagaaagcagaccacctccaagatcctggtgcggaacatccccttccaggcccacagccgggagatccgagagctcttcagcacctttggggagttgaagacggtccgcctgccaaagaagatgactgggacaggcacacacagaggcttcggctttgtggacttcctcaccaagcaggatgcgaagagagccttcaacgccctgtgtcacagcacccacttgtacgggcggaggctggtgctggagtgggccgactccgaggtgaccctgcaggccctgcggcggaagacggccgctcactttcacgagcccccgaagaaaaagcggtctgtggtgttggacgagatcctggagcagctggaaggcagtgacagcgacagcgaggagcagacccttcagctgtgagctggcaccgagaggggctgctgagctagaattcccacctatgtctttccaagggactgttcacggcttgggacttggtctctgtcctgccccatcctcgtcacttgggaccacgagccctggttcagtcacccagggaagcctcccagcggctcatgaagcatcgagctccaagcccagatgccaagctccctggctgagctgaatgatgtcactcatggtggacgcgttctgctcacgggcccagagccctgtgaaatgcatcaaggtcctctccgctggccagcagcatccccaggcttctctcaggcgcccgtgttcacattttctccagcctgagacgcagcctcccgcctggaagggcctgtgccagcaccaggcagagggcaagacggagagggcagagcaagaactgcactgcatctcactgcagtctgaatctagacatcgccattccccgaggtgcgacctcagactaatgacatcctggctgagcctctgtttttctctctaggaaatgggggtgataattgtgcctacctcagatagacagtgccagaattaagtgagtcaagccaagtaaagcccagagaagattctcatcaaaaaaaaaaaaaaaaaaaaaaaa

SEQ ID NO: 5 - protein, 960 aa, Homo sapiens [sequence omitted]